(19) United States
(12) Patent Application Publication
(10) Pub. No.: US 2007/0054849 A1
(43) Pub. Date: Mar. 8, 2007

(54) Method for diagnosing hepatocellular carcinomas

(75) Inventors: Yusuke Nakamura, Yokohama-shi (JP); Yoichi Furukawa, Kawasaki-shi (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Oncotherapy Science, Inc., Kanagawa (JP); The University of Tokyo, Tokyo (JP)

(21) Appl. No.: 10/572,932

(22) PCT Filed: Sep. 14, 2004
(86) PCT No.: PCT/JP04/13722

§ 371 Date: Nov. 9, 2006

Related U.S. Application Data
(60) Provisional application No. 60/505,632, filed on Sep. 24, 2003.

(51) Int. Cl. A61K 48/00; A61K 38/55; C12Q 1/68; G01N 33/567

(52) U.S. Cl. 514/12; 514/44; 435/6; 435/7.2

(57) ABSTRACT

Objective methods for detecting, diagnosing, treating and preventing hepatocellular carcinoma (HCC) are described herein. In particular, the present invention relates to two genes up-regulated in HCC cells as compared to normal cells, referred to herein as MGC47816 and HES6. In one embodiment, the diagnostic method involves the determining the expression level of MGC47816 or HES6 that discriminate between hepatocellular carcinoma cells and normal cells. The present invention further provides methods of screening for therapeutic agents useful in the treatment of HCC, methods of treating HCC, and methods for vaccinating a subject against HCC.

This application claims the benefit of U.S. Provisional Application Ser. No. 60/505,632 filed Sep. 24, 2003, the contents of which are hereby incorporated by reference in its entirety.

### FIELD OF THE INVENTION

The present invention relates to methods of detecting and diagnosing hepatocellular carcinomas as well as methods of treating and preventing same.

### BACKGROUND OF THE INVENTION

Hepatocellular carcinoma is one of the leading causes of cancer death worldwide. In spite of recent progress in diagnostic and therapeutic strategies, prognosis of patients with advanced cancer remains very poor. Although molecular studies have revealed that alterations of tumor suppressor genes and/or oncogenes are involved in carcinogenesis, the precise mechanisms remain unclear. In an effort to understand the mechanisms underlying tumor progression from a genome-wide point of view, to discover target molecules for diagnosis, and to develop novel therapeutic drugs, the present inventors have been analyzing gene expression profiles by means of a cDNA microarray representing 23,040 genes (Okabe et al., Cancer Res 61:2129-37 (2001); Kitahara et al., Cancer Res 61:3544-9) (2001); Lin et al., Oncogene 21:4120-8 (2002); Hasegawa et al., Cancer Res 62:7012-7 (2002)). In the course of these studies, a number of genes, including ESTs, which appear to be up-regulated frequently in the cancer tissues compared with the corresponding non-cancerous cells, have been identified. Since carcinogenesis involves activation of oncogenes and/or inactivation of tumor suppressor genes, enhanced expression of at least some of these up-regulated genes may reflect oncogenic properties.

cDNA microarray technologies have enabled comprehensive profiles of gene expression in normal and malignant cells to be obtained and compared (Okabe et al., Cancer Res 61:2129-37 (2001); Kitahara et al., Cancer Res 61: 3544-9 (2001); Lin et al., Oncogene 21:4120-8 (2002); Hasegawa et al., Cancer Res 62:7012-7 (2002)). This information assists in understanding the complex nature of cancer cells and the mechanisms of carcinogenesis. Identification of genes that are deregulated in tumors can lead to more precise and accurate diagnosis of individual cancers, and to the development of novel therapeutic targets (Bienz and Clevers, Cell 103:311-20 (2000)).

Studies designed to reveal mechanisms of carcinogenesis have already facilitated the identification of molecular targets for certain anti-tumor agents. For example, inhibitors of farnesyltransferase (FTIs) originally developed to inhibit the growth-signaling pathway related to Ras, whose activation depends on posttranslational farnesylation, have been shown to be effective in treating Ras-dependent tumors in animal models (He et al., Cell 99:335-45 (1999)). Similarly, clinical trials in humans using a combination of anti-cancer drugs and the anti-HER2 monoclonal antibody, trastuzumab, with the aim of antagonizing the proto-oncogene receptor HER2/neu have achieved improved clinical response and overall survival of breast-cancer patients (Lin et al., Cancer Res 61:6345-9 (2001)). Finally, a tyrosine kinase inhibitor, STI-571, which selectively inactivates bcr-abl fusion proteins, has been developed to treat chronic myelogenous leukemias wherein constitutive activation of bcr-ab1 tyrosine kinase plays a crucial role in the transformation of leukocytes. Agents of these kinds are designed to suppress oncogenic activity of specific gene products (Fujita et al., Cancer Res 61:7722-6 (2001)). Accordingly, it is apparent that gene products commonly up-regulated in cancerous cells may serve as potential targets for developing novel anti-cancer agents.

It has been further demonstrated that CD8+ cytotoxic T lymphocytes (CTLs) recognize epitope peptides derived from tumor-associated antigens (TAAs) presented on the MHC Class I molecule, and lyse tumor cells. Since the discovery of the MAGE family as the first example of TAAs, many other TAAs have been discovered using immunological approaches (Boon, Int J Cancer 54: 177-80 (1993); Boon and van der Bruggen, J Exp Med 183: 725-9 (1996); van der Bruggen et al., Science 254: 1643-7 (1991); Brichard et al., J Exp Med 178: 489-95 (1993); Kawakami et al., J Exp Med 180: 347-52 (1994)). Some of the newly discovered TAAs are currently undergoing clinical development as targets of immunotherapy. TAAs discovered so far include MAGE (van der Bruggen et al., Science 254: 1643-7 (1991)), gp100 (Kawakami et al., J Exp Med 180: 347-52 (1994)), SART (Shichijo et al., J Exp Med 187: 277-88 (1998)), and NY-ESO-1 (Chen et al., Proc Natl Acad Sci USA 94: 1914-8 (1997)). On the other hand, gene products demonstrated to be specifically overexpressed in tumor cells, have been shown to be recognized as targets inducing cellular immune responses. Such gene products include p53 (Umano et al., Brit J Cancer 84: 1052-7 (2001)), HER2/neu (Tanaka et al., Brit J Cancer 84: 94-9 (2001)), CEA (Nukaya et al., Int J Cancer 80: 92-7 (1999)), and so on.

In spite of significant progress in basic and clinical research concerning TAAs (Rosenbeg et al., Nature Med 4: 321-7 (1998); Mukherji et al., Proc Natl Acad Sci USA 92: 8078-82 (1995); Hu et al., Cancer Res 56: 2479-83 (1996)), only limited number of candidate TAAs for the treatment of adenocarcinomas, including hepatocellular carcinoma, are currently available. TAAs abundantly expressed in cancer cells, yet whose expression is restricted to cancer cells, would be promising candidates as immunotherapeutic targets. Further, identification of new TAAs inducing potent and specific antitumor immune responses is expected to encourage clinical use of peptide vaccination strategies for various types of cancer (Boon and can der Bruggen, J Exp Med 183: 725-9 (1996); van der Bruggen et al., Science 254: 1643-7 (1991); Brichard et al., J Exp Med 178: 489-95 (1993); Kawakami et al., J Exp Med 180: 347-52 (1994); Shichijo et al., J Exp Med 187: 277-88 (1998); Chen et al., Proc Natl Acad Sci USA 94: 1914-8 (1997); Harris, J Natl Cancer Inst 88: 1442-5 (1996); Butterfield et al., Cancer Res 59: 3134-42 (1999); Vissers et al., Cancer Res 59: 5554-9 (1999); van der Burg et al., J Immunol 156: 3308-14 (1996); Tanaka et al., Cancer Res 57: 4465-8 (1997); Fujie et al., Int J Cancer 80: 169-72 (1999); Kikuchi et al., Int J Cancer 81: 459-66 (1999); Oiso et al., Int J Cancer 81: 387-94 (1999)).

It has been repeatedly reported that peptide-stimulated peripheral blood mononuclear cells (PBMCs) from certain healthy donors produce significant levels of IFN-γ in response to the peptide, but rarely exert cytotoxicity against tumor cells in an HLA-A24 or -A0201 restricted manner in ⁵¹Cr-release assays (Kawano et al., Cancer Res 60: 3550-8 (2000); Nishizaka et al., Cancer Res 60: 4830-7 (2000); Tamura et al., Jpn J Cancer Res 92: 762-7 (2001)). However, both of HLA-A24 and HLA-A0201 are popular HLA alleles in the Japanese, as well as the Caucasian populations (Date et al., Tissue Antigens 47: 93-101 (1996); Kondo et al., J Immunol 155: 4307-12 (1995); Kubo et al., J Immunol 152: 3913-24 (1994); Imanishi et al., Proceeding of the eleventh International Histocompatibility Workshop and Conference Oxford University Press, Oxford, 1065 (1992); Williams et al., Tissue Antigen 49: 129 (1997)). Thus, antigenic peptides of carcinomas presented by these HLAs may be especially useful for the treatment of carcinomas among Japanese and Caucasians. Further, it is known that the induction of low-affinity CTL in vitro usually results from the use of peptide at a high concentration, generating a high level of specific peptide/MHC complexes on antigen presenting cells (APCs), which will effectively activate these CTL (Alexander-Miller et al., Proc Natl Acad Sci USA 93: 4102-7 (1996)).

Accordingly, to disclose mechanisms of hepatocellular carcinogenesis and to identify novel diagnostic markers and molecular targets for anticancer drugs for hepatocellular carcinoma (HCC), expression profiles of 20 HCCs were analyzed using a genome-wide cDNA microarray containing 23,040 genes. Among the genes with altered expression in the tumors, two human genes, MGC47816 and HES6, frequently up-regulated in the cancers compared with the corresponding normal tissues were selected. The one gene, MGC47816, encoded a putative 391-amino-acid protein containing a carbamoyl-phosphate synthase L chain and an ATP binding domain, and was assigned at chromosomal band 1q34.1. The other gene, HES6, encoded a putative 224-amino-acid protein containing a helix-loop-helix domain and orange domain, and was assigned at chromosomal band 2q37.Suppressed expression of MGC47816 or HES6 by transfection of short interfering RNA (siRNA) inhibited the growth of hepatocellular carcinoma cells. These results provide novel insight into hepatocellular carcinogenesis, and may contribute to the development of new strategies for diagnosis and treatment of this cancer.

### SUMMARY OF THE INVENTION

Accordingly, the present invention is based on the discovery of a pattern of gene expression of MGC47816 and HES6 that correlate with hepatocellular carcinoma (HCC).

Accordingly, the present invention provides a method of detecting, diagnosing and/or determining a predisposition to HCC in a subject by determining an expression level of MGC47816 or HES6 in a patient-derived biological sample, such as tissue sample, and comparing it to a control expression level. An increase in the expression level of MGC47816 or HES6 as compared to a normal control level of the gene indicates that the subject suffers from or is at risk of developing HCC.

In the context of the present invention, the phrase “control level” refers to an expression level detected in a control sample and includes both a normal control level and an HCC control level. In the context of the present invention, a control level may comprise a single expression pattern derived from a single reference population or from a plurality of expression patterns. For example, the control level can be a database of expression patterns from previously tested cells. A “normal control level” refers to a level of gene expression detected in a normal individual or in a population of individuals known not to be suffering from HCC. A normal individual is one with no clinical symptoms of HCC. A normal cell is preferably obtained from hepatocellular tissue. On the other hand, an “HCC control level” refers to a level of gene expression detected in an individual or population of individuals known to be suffering from HCC.

An increase in the expression level MGC47816 or HES6 detected in a test sample as compared to a normal control level indicates that the subject (from which the sample was obtained) suffers from or is at risk of developing HCC.

According to the present invention, an expression level is deemed “increased” when gene expression is increased by at least 10%, at least 25%, or at least 50% or more as compared to a control level. Alternatively, an expression level is deemed “increased” when gene expression is increased at least 0.1, at least 0.2, at least 1, at least 2, at least 5, or at least 10 or more fold as compared to a control level. Expression can be determined by detecting hybridization, e.g., binding of an MGC47816 or HES6 gene probe to a gene transcript isolated from a patient-derived tissue sample.

In the context of the present invention, the patient-derived tissue sample may be any tissue taken from a test subject, e.g., a patient known to or suspected of having HCC. For example, the tissue may contain a liver cancer cell. More particularly, the tissue may be a cell from liver.

The present invention further provides methods of identifying an agent that inhibits the expression of MGC47816 or HES6 or the activity of their gene products by contacting a test cell expressing MGC47816 or HES6 with a test agent and determining the expression level or activity of the MGC47816 or HES6 gene or gene product, respectively. The test cell is preferably a hepatocellular cell, such as a hepatocellular cell from a hepatocellular carcinoma. A decrease in the expression level of MGC47816 or HES6 as compared to a normal control level of the gene indicates that the test agent is an inhibitor of MGC47816 or HES6 and, therefore, reduces a symptom of HCC.

The invention also provides a kit comprising a detection reagent which binds to MGC47816 or HES6 nucleic acid sequences or to a gene product encoded thereby.

Therapeutic methods of the present invention include a method of treating or preventing HCC in a subject including the step of administering to the subject an antisense composition. In the context of the present invention, the antisense composition reduces the expression of the specific target gene, e.g., MGC47816 or HES6. For example, the antisense composition may contain a nucleotide, which is complementary to a nucleic acid sequence of MGC47816 or HES6. Alternatively, the present method may includes the step of administering to a subject an small interfering RNA (siRNA) composition. In the context of the present invention, the siRNA composition reduces the expression of MGC47816 or HES6.

In yet another embodiment, the present invention provides a method of treating or preventing of HCC in a subject including the step of administering to a subject a ribozyme composition. The nucleic acid-specific ribozyme composition reduces the expression of MGC47816 or HES6. Suitable mechanisms for in vivo expression of a gene of interest are known in the art.

The invention also provides vaccines and vaccination methods. For example, a method of treating or preventing HCC in a subject may involve administering to the subject a vaccine containing a polypeptide encoded by MGC47816 or HES6 or an immunologically active fragment such a polypeptide. In the context of the present invention, an immunologically active fragment is a polypeptide that is shorter in length than the full-length naturally-occurring protein yet which induces an immune response analogous to that induced by the full-length protein. For example, an immunologically active fragment should be at least 8 residues in length and capable of stimulating an immune cell such as a T cell or a B cell. Immune cell stimulation can be measured by detecting cell proliferation, elaboration of cytokines (e.g., IL-2), or production of an antibody.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference herein in their entirety. In case of conflict, the present specification, including definitions, will control. In addition, the materials, methods, and examples are illustrative only and not intended to be limiting.

One advantage of the methods described herein is that the disease is identified prior to detection of overt clinical symptoms. Other features and advantages of the invention will be apparent from the following detailed description, and from the appended claims.

### BRIEF DESCRIPTION OF THE DRAWINGS

FIG. 1 depicts relative expression ratios (cancer/non-cancer) of D4999 in 20 primary HCCs examined by cDNA microarray. Up-regulated expression (Cy3:Cy5 intensity ratio, >2.0) was observed in 7 of the 11 HCCs that passed through the cutoff filter (both Cy3 and Cy5 signals greater than 25,000).

FIG. 2 depicts the expression of D4999 analyzed by semi-quantitative RT-PCR using additional HCC tissues. T refers to tumor tissue; N, to normal tissue. Expression of GAPDH served as an internal control.

FIG. 3 depicts the genomic structure of MGC47816 and the predicted structure of the MGC47816 protein. Exons are indicated by open boxes with nucleotide numbers of MGC47816 cDNA shown in the upper panel.

FIG. 4 depicts the subcellular localization of HA-tagged MGC47816 protein. Immunoblotting of HA-tagged MGC47816 protein is shown in FIG. 4(a). Immunohistochemical staining of the tagged proteins in COS7 cells is shown in FIG. 4(b). The protein was stained with rat anti-HA monoclonal antibody and visualized by RHODAMINE-conjugated secondary anti-rat IgG antibody. Nuclei were counter-stained with DAPI.

FIG. 5 depicts the effect of MGC47816-siRNA on the expression of MGC47816 [FIG. 5(a)] and the effect of MGC47816-siRNA on the viability of Alexander and SNU449 cells [FIG. 5(b)].

FIG. 6(a) depicts relative expression ratios (cancer/non-cancer) of C2298 in 20 primary HCCs examined by cDNA microarray. Up-regulated expression (Cy3:Cy5 intensity ratio, >2.0) was observed in 11 of the 12 HCCs that passed through the cutoff filter (both Cy3 and Cy5 signals greater than 25,000). FIG. 6(b) depicts the expression of C2298, analysed by semi-quantitative RT-PCR, in eight additional HCCs (T) and their corresponding non-cancerous liver tissues (N). Expression of GAPDH served as an internal control.

FIG. 7 depicts the results of multi-tissue Northern blot analysis of HES6. The transcript of HES6 is approximately 1.4-kb by size.

FIG. 8 depicts the genomic structure of HES6 and the predicted structure of the HES6 protein. Exons are indicated by open boxes with nucleotide numbers of HES6 cDNA shown in the upper panel.

FIG. 9 depicts the subcellular localization of tagged HES6 protein. FIG. 9(a) depicts the results of immunoblotting of HA-tagged HES6 protein. FIG. 9(b) depicts the results of immunohistochemical staining of the tagged protein in COS7 cells. HA-tagged HES6 protein was stained with rat anti-HA monoclonal antibody and visualized by RHODAMINE-conjugated secondary anti-rat IgG antibody. Nuclei were counter-stained with DAPI.

FIG. 10 depicts effects of HES6-siRNA on the expression of HES6 [FIG. 10(a)] and the effect of HES6-siRNA on the viability of Alexander and HepG2 cells (b).

### DETAILED DESCRIPTION OF THE INVENTION

The words “a”, “an” and “the” as used herein mean “at least one” unless otherwise specifically indicated.

The present invention is based, in part, on the discovery of elevated expression of MGC47816 and HES6 in the liver cells of patients with HCC. This elevated gene expression was identified using a comprehensive cDNA microarray system.

Using a cDNA microarray containing 23,040 genes, comprehensive gene-expression profiles of 20 patients were previously constructed. MGC47816 and HES6 are expressed at high level in HCC patients. Candidate molecular markers having the potential to detect cancer-related proteins in serum or sputum of patients were selected, and some potential targets for development of signal-suppressing strategies in human hepatocellular carcinoma were discovered. In particular, MGC47816 and HES6 are identified herein as markers of HCC having diagnostic utility and as gene targets, the expression of which may be altered to treat or alleviate a symptom of HCC.

Unless indicated otherwise, “HCC” refers to hepatocellular carcinoma and an HCC-associated gene or protein refers to any of the nucleic or amino acid sequences disclosed herein (e.g., MGC47816 or HES6).

By measuring expression of MGC47816 or HES6 in a sample of cells, HCC can be diagnosed. Similarly, by measuring the expression of MGC47816 or HES6 in response to various agents, and agents for treating HCC can be identified.

The present invention involves determining (e.g., measuring) the expression of MGC47816 or HES6. Using sequence information provided by the GeneBank™ database entries for the MGC47816 and HES6 nucleotide and/or amino acid sequences, respectively, MGC47816 or HES6 can be detected and measured using techniques well known to one of ordinary skill in the art. For example, a sequence within the sequence database entries corresponding to MGC47816 or HES6, can be used to construct probes for detecting MGC47816 or HES6 RNA sequence using, e.g., Northern blot hybridization analysis. As another example, the published sequences can be used to construct primers for specifically amplifying MGC47816 or HES6 using, e.g., amplification-based detection methods, such as reverse-transcription based polymerase chain reaction.

The expression level of MGC47816 or HES6 in a test cell population, e.g., a patient-derived tissues sample, is then compared to the expression level of MGC47816 or HES6 in a reference population. The reference cell population includes one or more cells for which the compared parameter is known, i.e., HCC cells or non-HCC cells.

Whether or not a pattern of gene expression in the test cell population compared to the reference cell population indicates HCC or a predisposition thereto depends upon the composition of the reference cell population. For example, if the reference cell population is composed of non-HCC cells, a similar gene expression pattern between the test cell population and the reference cell population indicates the test cell population is non-HCC. Conversely, if the reference cell population is made up of HCC cells, a similar gene expression profile between the test cell population and the reference cell population indicates that the test cell population includes HCC cells.

A level of expression of an HCC marker gene in a test cell population is considered “altered” if it varies from a level of expression associated with a reference cell population by more than 1.2, more than 1.5, more than 2.0, more than 5.0, or more than 10.0 or more fold.

Differential gene expression between a test cell population and a reference cell population can be normalized to a control nucleic acid, e.g., a housekeeping gene. In the context of the present invention, a control nucleic acid is one whose expression is known not to vary between cancerous and non-cancerous states of the cell. Expression levels of a control nucleic acid in the test and reference nucleic acid can be used to normalize signal levels in the compared populations. Examples of control genes include, but are not limited to, β-ctin, glyceraldehyde 3-phosphate dehydrogenase and ribosomal protein P1.

A test cell population may be compared to multiple reference cell populations. Each of the multiple reference populations may differ in the known parameter. Thus, a test cell population may be compared to a first reference cell population known to contain, e.g., HCC cells, as well as a second reference population known to contain, e.g., non-HCC cells (normal cells). The test cell is isolated from a tissue type or cell sample taken from a subject known to contain, or to be suspected of containing, HCC cells.

The test cell is obtained from a bodily tissue or a bodily fluid, e.g., biological fluid (such as blood or urine). For example, the test cell can be purified from a tissue. Preferably, the test cell population comprises an epithelial cell. More preferably, the epithelial cell is from a tissue known to be or suspected to be an HCC.

Cells in the reference cell population should be derived from a tissue type as similar to test cell. Optionally, the reference cell population is a cell line, e.g., an HCC cell line (positive control) or a normal, non-HCC cell line (negative control). Alternatively, the control cell population can be derived from a database of molecular information derived from cells for which the assayed parameter or condition is known.

The subject is preferably a mammal. The mammal can be, e.g., a human, non-human primate, mouse, rat, dog, cat, horse, or cow.

Expression of MGC47816 or HES6 can be determined at the protein or nucleic acid level, using methods known in the art. For example, Northern hybridization analysis, using probes which specifically recognize an RNA sequence associated with MGC47816 or HES6, can be used to determine gene expression. Alternatively, gene expression can be measured using reverse-transcription-based PCR assays, e.g., using primers specific for MGC47816 or HES6. Expression can also be determined at the protein level, i.e., by measuring the levels of polypeptide encoded by an HCC marker genes described herein, or the biological activity thereof. Such methods are well known in the art and include, but are not limited to, e.g., immunoassays based on antibodies to protein encoded by MGC47816 or HES6. The biological activities of the proteins encoded by the respective genes are also well known. For example, recent studies suggest that human HES6 inhibits and promotes the proteolytic degradataion of HES 1, supports MASH1 activity and promotes cell, particularly myogenic and neuronal cell, differentiation (Bae S, et al., Development. July 2000; 127(13):293343; Gao X et al., J Cell Biol. Sep. 17, 2001; 154(6):1161-71).

Diagnosing HCC:

In the context of the present invention, HCC is diagnosed by measuring the expression level of MGC47816 or HES6 in a test population of cells, (i.e., a patient-derived biological sample). Preferably, the test cell population contains an epithelial cell, e.g., a cell obtained from liver tissue. Gene expression can also be measured from blood or other bodily fluids, such as urine. Other biological samples can be used to determine protein level. For example, the level of protein in blood or serum derived from a subject to be diagnosed can be measured by immunoassay or other conventional biological assays.

Expression of MGC47816 or HES6 is determined in the test cell or biological sample and compared to expression level associated with a normal control sample. A normal control level is an expression profile of MGC47816 or HES6 typically found in a population known not to be suffering from HCC. Accordingly, an increase in the level of expression of MGC47816 or HES6 in a patient-derived tissue sample indicates that the subject is suffering from or is at risk of developing HCC.

In other words, when the level of expression of MGC47816 or HES6 is altered in a test population as compared to a normal control, this indicates that the test subject suffers from or is at risk of developing HCC.

Identifying Agents That Inhibit MGC47816 or HES6 Expression or Activity:

An agent that inhibits the expression of MGC47816 or HES6 or the activity of a gene product associated therewith can be identified by contacting a test cell population expressing MGC47816 or HES6 with a test agent and determining the expression level of MGC47816 or HES6 or the activity of gene product associated therewith. A decrease in expression or activity in the presence of the agent as compared to the level in the absence of the test agent indicates that the agent is an inhibitor of MGC47816 or HES6 and, therefore, may be useful in inhibiting HCC.

The test cell population can be any cell expressing MGC47816 or HES6. For example, the test cell population may contain an epithelial cell, such as a cell isolated or derived from liver. In particular, the test cell may be an immortalized cell line derived from hepatocellular carcinoma. Alternatively, the test cell may be a cell transfected with MGC47816 or HES6 or which has been transfected with a regulatory sequence (e.g. promoter sequence) from MGC47816 or HES6 operably linked to a reporter gene.

Assessing Efficacy of Treatment of HCC in a Subject:

The differentially expressed MGC47816 or HES6 identified herein also allow for the course of treatment of HCC to be monitored. In this method, a test cell population is provided from a subject undergoing treatment for HCC. If desired, test cell populations can be obtained from the subject at various time points before, during, and/or after treatment. Expression of MGC47816 or HES6 in the cell population is then determined and compared to a reference cell population, which includes cells whose HCC state is known. In the context of the present invention, the reference cells should not have been exposed to the treatment of interest.

If the reference cell population contains no HCC cells, a similarity in the expression of MGC47816 or HES6 between a test cell population and a normal control reference cell population indicates that the treatment is efficacious. However, a difference in expression of MGC47816 or HES6 between a test population and a normal control reference cell population indicates the less favorable clinical outcome or prognosis. Conversely, if the reference cell population contains HCC cells, a difference in the expression of an HCC-associated gene (e.g., MGC47816 or HES6) between a test cell population and the reference cell population indicates that the treatment of interest is efficacious, while a similarity in the expression of MGC47816 or HES6 in a test population and a reference cell population indicates a less favorable clinical outcome or prognosis.

Additionally, the expression level of one or more HCC-associated genes (e.g., MGC47816 or HES6) determined in a subject-derived biological sample obtained after treatment (i.e., post-treatment levels) can be compared to the expression level of the one ore more HCC-associated genes determined in a subject-derived biological sample obtained prior to treatment onset (i.e., pre-treatment levels). A decrease in the expression of MGC47816 and/or HES6 in a post-treatment sample indicates that the treatment of interest is efficacious, while an increase or maintenance in expression in the post-treatment sample indicates a less favorable clinical outcome or prognosis. In the context of the present invention, the term “efficacious indicates that the treatment leads to a reduction in the expression of a pathologically up-regulated gene, or a decrease in size, prevalence, or metastatic potential of hepatocellular tumors in a subject. When a treatment of interest is applied prophylactically, the term “efficacious” means that the treatment retards or prevents HCC from forming or retards, prevents, or alleviates a symptom of clinical HCC. Assessment of hepatocellular tumors can be made using standard clinical protocols.

In addition, efficaciousness can be determined in association with any known method for diagnosing or treating HCC. For example, HCC can be diagnosed by identifying symptomatic anomalies.

Selecting a Therapeutic Agent for Treating HCC That is Appropriate for a Particular Individual:

Differences in the genetic makeup of individuals can result in differences in their relative abilities to metabolize various drugs. An agent that is metabolized in a subject to act as an anti-HCC agent can manifest itself by inducing a change in a gene expression pattern in the subject's cells from that characteristic of an HCC state to a gene expression pattern characteristic of a non-HCC state. Accordingly, the differentially expressed MGC47816 or HES6 genes disclosed herein allow for a putative therapeutic for HCC or a prophylactic inhibitor of HCC to be tested in a test cell population from a selected subject to determine if the agent is a suitable inhibitor of HCC in the subject.

To identify an inhibitor of HCC that is appropriate for a specific subject, a test cell population from the subject is exposed to a therapeutic agent, and the expression of MGC47816 or HES6 is determined.

In the context of the present invention, the test cell population contains an HCC cell expressing MGC47816 or HES6. Preferably, the test cell is an epithelial cell. For example, a test cell population may be incubated in the presence of a candidate agent. Next, the pattern of gene expression in the test sample is measured and compared to one or more reference profiles, e.g., an HCC reference expression profile or a non-HCC reference expression profile.

A decrease in the expression of MGC47816 or HES6 in a test cell population relative to a reference cell population containing HCC indicates that the agent is therapeutic.

The test agent can be any compound or composition. Exemplary test agents suitable for use in the present invention include, but are not limited to, immunomodulatory agents.

Screening Assays for Identifying Therapeutic Agents:

MGC47816 or HES6 disclosed herein can also be used to identify candidate therapeutic agents for treating HCC. The method of the present invention involves the step of screening a candidate therapeutic agent to determine if it converts an expression profile of MGC47816 or HES6 characteristic of an HCC state to a pattern indicative of a non-HCC state.

In the instant method, a cell is exposed to a test agent or a plurality of test agents (sequentially or in combination) and the expression of MGC47816 or HES6 in the cell is measured. The expression level of MGC47816 or HES6 in the test population is then compared to the expression level of MGC47816 or HES6 in a reference cell population that has not been exposed to the test agent.

An agent capable of suppressing the expression of a gene over-expressed in HCC (e.g., MGC47816 or HES6) has potential clinical benefit. Such compounds can be further tested for the ability to prevent HCC growth.

In a further embodiment, the present invention provides methods for screening candidate agents which are potential targets in the treatment of HCC. As discussed in detail above, by controlling the expression level of a marker gene or the activity of its gene product, one can control the onset and progression of HCC. Thus, candidate agents, which are potential targets in the treatment of HCC, can be identified through screening methods that use such expression levels and activities as indices of the cancerous or non-cancerous state.

Accordingly, in the context of the present invention, such screening may comprise, for example, the following steps:
a) contacting a test compound with a polypeptide encoded by MGC47816 or HES6;
b) detecting the binding activity between the polypeptide and the test compound; and
c) selecting the test compound that binds to the polypeptide

Alternatively, the screening method of the present invention may comprise the following steps:
a) contacting a candidate compound with a cell expressing MGC47816 or HES6, and
b) selecting the candidate compound that reduces the expression level of MGC47816 or HES6.

Cells expressing marker gene(s) include, for example, cell lines established from HCC; such cells can be used for the above screening of the present invention.

Alternatively, the screening method of the present invention may comprise the following steps:
a) contacting a test compound with a polypeptide encoded by MGC47816 or HES6;
b) detecting the biological activity of the polypeptide of step (a); and
c) selecting the test compound that suppresses the biological activity of the polypeptide encoded by MGC47816 or HES6 as compared to the biological activity of said polypeptide detected in the absence of the test compound.

A protein for use in the screening methods of the present invention can be obtained as a recombinant protein using the nucleotide sequence of the marker gene. Based on the information regarding the marker gene and/or its encoded protein, one skilled in the art can select any biological activity of the protein as an index for screening and any suitable measurement method to assay for the selected biological activity. Preferably, the cell proliferative activity of MGC47816 or HES6 is selected as the biological activity. Cell proliferative activity can be routinely detected by proliferation of cell lines, such as NIH3T3 or COS7.

Alternatively, the screening method of the present invention may comprise the following steps:
a) contacting a candidate compound with a cell into which a vector, comprising the transcriptional regulatory region of MGC47816 or HES6 and a reporter gene that is expressed under the control of the transcriptional regulatory region, has been introduced
b) measuring the expression or activity of said reporter gene; and
c) selecting the candidate compound that reduces the expression or activity of said reporter gene, as compared to a control.

Suitable reporter genes and host cells are well known in the art. A reporter construct for use in the screening method of the present invention can be prepared by using the transcriptional regulatory region of an HCC-associated marker gene (e.g., MGC47816 or HES6). When the transcriptional regulatory region of a marker gene is known to those skilled in the art, a reporter construct can be prepared by using previous sequence information. When the transcriptional regulatory region of a marker gene remains unidentified, a nucleotide segment containing the transcriptional regulatory region can be isolated from a genome library based on the nucleotide sequence information of the marker gene.

A compound isolated by the screening can serve as a candidate for the development of drugs that inhibit the activity of the protein encoded by the marker gene and can be applied to the treatment or prevention of HCC.

Moreover, compounds in which a part of the structure of the compound inhibiting the activity of protein encoded by the marker gene is converted by addition, deletion and/or replacement are also included as compounds obtainable by the screening method of the present invention.

When administrating a compound isolated by the method of the present invention as a pharmaceutical for humans and other mammals, such as mice, rats, guinea-pigs, rabbits, cats, dogs, sheep, pigs, cattle, monkeys, baboons, and chimpanzees, the isolated compound can be directly administered or can be formulated into a dosage form using known pharmaceutical preparation methods. For example, according to the need, the drugs can be taken orally, as sugar-coated tablets, capsules, elixirs and microcapsules, or non-orally, in the form of injections of sterile solutions or suspensions with water or any other pharmaceutically acceptable liquid. For example, the compounds can be mixed with pharmaceutically acceptable carriers or media, specifically, sterilized water, physiological saline, plant-oils, emulsifiers, suspending agents, surfactants, stabilizers, flavoring agents, excipients, vehicles, preservatives, binders, and such, in a unit dose form required for generally accepted drug implementation. The amount of active ingredient contained in such a preparation makes a suitable dosage within the indicated range acquirable.

Examples of additives that can be admixed into tablets and capsules include, but are noted limited to, binders, such as gelatin, corn starch, tragacanth gum and arabic gum; excipients, such as crystalline cellulose; swelling agents, such as corn starch, gelatin and alginic acid; lubricants, such as magnesium stearate; sweeteners, such as sucrose, lactose or saccharin; and flavoring agents, such as peppermint, Gaultheria adenothrix oil and cherry. When the unit-dose form is a capsule, a liquid carrier, such as an oil, can also be further included in the above ingredients. Sterile composites for injections can be formulated following normal drug implementations using vehicles such as distilled water suitable for injection.

Physiological saline, glucose, and other isotonic liquids, including adjuvants, such as D-sorbitol, D-mannnose, D-mannitol, and sodium chloride, can be used as aqueous solutions for injection. These can be used in conjunction with suitable solubilizers, such as alcohol, for example, ethanol; polyalcohols, such as propylene glycol and polyethylene glycol; and non-ionic surfactants, such as Polysorbate 80 (TM) and HCO-50.

Sesame oil or soy-bean oil can be used as an oleaginous liquid, may be used in conjunction with benzyl benzoate or benzyl alcohol as a solubilizer, and may be formulated with a buffer, such as phosphate buffer and sodium acetate buffer; a pain-killer, such as procaine hydrochloride; a stabilizer, such as benzyl alcohol and phenol; and/or an anti-oxidant. A prepared injection may be filled into a suitable ampoule.

Methods well known to those skilled in the art may be used to administer the pharmaceutical composition of the present invention to patients, for example as an intraarterial, intravenous, or percutaneous injection or as an intranasal, transbronchial, intramuscular or oral administration. The dosage and method of administration vary according to the body-weight and age of a patient and the administration method; however, one skilled in the art can routinely select a suitable method of administration. If said compound is encodable by a DNA, the DNA can be inserted into a vector for gene therapy and the vector administered to a patient to perform the therapy. The dosage and method of administration vary according to the body-weight, age, and symptoms of the patient; however, one skilled in the art can suitably select them.

For example, although the dose of a compound that binds to a protein of the present invention and regulates its activity depends on the symptoms, the dose is generally about 0.1 mg to about 100 mg per day, preferably about 1.0 mg to about 50 mg per day and more preferably about 1.0 mg to about 20 mg per day, when administered orally to a normal adult human (weight 60 kg).

When administering parenterally, in the form of an injection to a normal adult human (weight 60 kg), although there are some differences according to the patient, target organ, symptoms and method of administration, it is convenient to intravenously inject a dose of about 0.01 mg to about 30 mg per day, preferably about 0.1 to about 20 mg per day and more preferably about 0.1 to about 10 mg per day. Also, in the case of other animals, i the appropriate dosage amount may be routinely calculated by converting to 60 kgs of body-weight.

Assessing the Prognosis of a Subject with HCC:

The present invention also provides a method of assessing the prognosis of a subject with HCC, including the step of comparing the expression of MGC47816 or HES6 in a test cell population to the expression of the gene in a reference cell population derived from patients over a spectrum of disease stages. By comparing gene expression of MGC47816 or HES6 in the test cell population and the reference cell population(s), or by comparing the pattern of gene expression over time in test cell populations derived from the subject, the prognosis of the subject can be assessed.

For example, an increase in the expression of MGC47816 or HES6 in a test cell as compared to a normal control indicates less favorable prognosis. Conversely, a similarity in the expression of MGC47816 or HES6 between a test cell and a normal control indicates a more favorable prognosis for the subject.

Kits:

The present invention also includes an HCC-detection reagent, e.g., a nucleic acid that specifically binds to or identifies an MGC47816 or HES6 nucleic acid, such as oligonucleotide sequences which are complementary to a portion of an MGC47816 or HES6 nucleic acid or antibodies that bind to proteins encoded by an MGC47816 or HES6 nucleic acid. The reagents may be packaged together in the form of a kit. For example, the reagents may be packaged in separate containers, e.g., a nucleic acid or antibody (either bound to a solid matrix or packaged separately with reagents for binding it to the matrix) in one container, a control reagent (positive and/or negative) in a second container, and/or a detectable label in a third container. Instructions (e.g., written, tape, CD-ROM, etc.) for carrying out the assay may also be included in the kit. The assay format of the kit may be a Northern hybridization or a sandwich ELISA, both of which known in the art.

For example, an HCC detection reagent may be immobilized on a solid matrix, such as a porous strip, to form at least one HCC detection site. The measurement or detection region of the porous strip may include a plurality of sites, each containing a nucleic acid. A test strip may also contain sites for negative and/or positive controls. Alternatively, control sites may be located on a separate strip from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of HCC present in the sample. The detection sites may be configured in any suitably detectable shape and are typically in the shape of a bar or dot spanning the width of a test strip.

Methods of Inhibiting HCC:

The present invention further provides a method for treating or alleviating a symptom of HCC in a subject by decreasing expression of an HCC-associated gene (e.g., MGC47816 or HES6) or an activity of one of their gene products. Suitable therapeutic compounds can be administered prophylactically or therapeutically to subject suffering from or at risk of (or susceptible to) developing HCC. Administration can be systemic or local. Such subjects can be identified using standard clinical methods or by detecting an aberrant level of expression of MGC47816 or HES6 or activity of one of their gene products. Exemplary therapeutic agents include, but are not limited to, inhibitors of cell proliferation.

The therapeutic method of the present invention includes decreasing the expression of MGC47816 or HES6, the function of one of their gene products, or both. Expression may be inhibited in any of several ways known in the art. For example, expression can be inhibited by administering to the subject a nucleic acid that inhibits, or antagonizes, the expression of the over-expressed gene, e.g., an antisense oligonucleotide or small interfering RNA which disrupts expression of the over-expressed gene.

Antisense Nucleic Acids:

As noted above, antisense nucleic acids corresponding to the nucleotide sequence of MGC47816 or HES6 can be used to reduce the expression level of MGC47816 or HES6. Antisense nucleic acids corresponding to the nucleotide sequence of genes that are up-regulated in HCC (e.g., MGC47816 or HES6) are useful in the treatment of HCC. Specifically, antisense nucleic acids of the present invention may act by binding to the nucleotide sequence of MGC47816 or HES6 or an mRNA corresponding thereto, thereby inhibiting the transcription or translation of the gene, promoting the degradation of the mRNA, and/or inhibiting the expression of a protein encoded by an MGC47816 or HES6 nucleic acid, and finally inhibiting the function of such a protein. The term “antisense nucleic acids” as used herein encompasses both nucleotides that are entirely complementary to a target sequence and those having a mismatch of nucleotide, so long as the antisense nucleic acids can specifically hybridize to the target sequences. For example, antisense nucleic acids of the present invention include polynucleotides having a homology to a reference sequence of at least 70% or higher, preferably at least 80% or higher, more preferably at least 90% or higher, even more preferably at least 95% or higher, over a span of at least 15 continuous nucleotides. Algorithms known in the art can be used to determine homology.

The antisense nucleic acids of the present invention act on cells producing the protein encoded by an HCC-associated marker gene by binding to the DNA or mRNA encoding the protein, inhibiting transcription or translation, promoting the degradation of the mRNA, and/or inhibiting the expression of the protein, thereby resulting in inhibition of the protein function.

An antisense nucleic acid of the present invention can be made into an external preparation, such as a liniment or a poultice, by admixing it with a suitable base material which is inactive against the nucleic acid.

Also, as needed, the antisense nucleic acids can be formulated into tablets, powders, granules, capsules, liposome capsules, injections, solutions, nose-drops, freeze-drying agents, and the like, by adding excipients, isotonic agents, solubilizers, stabilizers, preservatives, pain-killers, and such. These can be prepared by known methods.

For example, an antisense nucleic acid of the present invention can be given to a patient by direct application onto the ailing site or by injection into a blood vessel so that it will reach the site of ailment. An antisense-mounting medium can also be used to increase durability and membrane-permeability. Examples include, but are not limited to, liposomes, poly-L-lysine, lipids, cholesterol, lipofectin or derivatives of these.

The dosage of the antisense nucleic acid of the present invention can be adjusted suitably according to the patient's condition and used in desired amounts. For example, a dose range of 0.1 to 100 mg/kg, preferably 0.1 to 50 mg/kg can be administered.

The antisense nucleic acids of the present invention inhibit the expression of a protein of the invention and are thereby useful for suppressing the biological activity of the protein. In addition, expression-inhibitors, comprising antisense nucleic acids of the invention, are useful in that they can inhibit the biological activity of a protein of the present invention.

The antisense nucleic acids of present invention include modified oligonucleotides. For example, thioated nucleotides may be used to confer nuclease resistance to an oligonucleotide.

Also, a siRNA against marker gene can be used to reduce the expression level of the marker gene. By the term “siRNA” is meant a double stranded RNA molecule which prevents translation of a target mRNA. Standard techniques of introducing siRNA into the cell are used, including those in which DNA is a template from which siRNA is transcribed. In the context of the present invention, the siRNA comprises a sense nucleic acid sequence and an anti-sense nucleic acid sequence against an upregulated marker gene, such as MGC47816 or HES6. The antisense and siRNA method of the present invention can be used to alter the expression in a cell of an up-regulated HCC gene, e.g., up-regulation resulting from the malignant transformation of the cells. Binding of an siRNA to a transcript corresponding to MGC47816 or HES6 in the target cell results in a reduction in the protein production by the cell. The length of the oligonucleotide is at least 10 nucleotides and may be as long as the naturally-occurring the transcript. Preferably, the oligonucleotide is about 19-25 nucleotides in length. Most preferably, the oligonucleotide is less than 75, less than 50, or less than 25 nucleotides in length. Examples of MGC47816 siRNA oligonucleotides which inhibited the expression in Alexander and SNU449 cells include the target sequence containing SEQ ID NO: 19. Examples of HES6 siRNA oligonucleotides which inhibited the expression in Alexander and HepG2 cells include the target sequence containing SEQ ID NO: 26.

The siRNA can be constructed such that a single transcript has both the sense and complementary antisense sequences from the target gene, e.g., as a hairpin.

An siRNA of an HCC-associated gene (e.g., MGC47816 or HES6) hybridizes to target mRNA and thereby decreases or inhibits production of the MGC47816 or HES6 polypeptides by associating with the normally single-stranded mRNA transcript, thereby interfering with translation and thus, expression of the protein. In order to enhance the inhibition activity of an siRNA, nucleotide “u” can be added to 3′end of the antisense strand of the target sequence. The number of “u”s to be added is at least 2, generally 2 to 10, preferably 2 to 5. The added “u”s form single strand at the 3′end of the antisense strand of the siRNA.

An siRNA of MGC47816 or HES6 can be directly introduced into the cells in a form that is capable of binding to the mRNA transcripts. Alternatively, a DNA encoding the siRNA may be carried in a vector.

Vectors may be produced, for example, by cloning an HCC-associated gene target sequence into an expression vector having operatively-linked regulatory sequences flanking the sequence in a manner that allows for expression (by transcription of the DNA molecule) of both strands (Lee, N. S., Dohjima, T., Bauer, G., Li, H., Li, M.-J., Ehsani, A., Salvaterra, P., and Rossi, J. (2002) Expression of small interfering RNAs targeted against HIV-1 rev transcripts in human cells. Nature Biotechnology 20: 500-505.). An RNA molecule that is antisense to mRNA of an HCC-associated gene (e.g., MGC47816 or HES6) is transcribed by a first promoter (e.g., a promoter sequence 3′ of the cloned DNA) and an RNA molecule that is the sense strand for the mRNA of the HCC-associated gene is transcribed by a second promoter (e.g., a promoter sequence 5′ of the cloned DNA). The sense and antisense strands hybridize in vivo to generate siRNA constructs for silencing of the HCC-associated gene. Alternatively, the two constructs can be utilized to create the sense and anti-sense strands of a siRNA construct. Cloned HCC-associated genes (e.g., MGC47816 or HES6) can encode a construct having secondary structure, e.g., hairpins, wherein a single transcript has both the sense and complementary antisense sequences from the target gene.

A loop sequence consisting of an arbitrary nucleotide sequence can be located between the sense and antisense sequence in order to form the hairpin loop structure. Thus, the present invention also provides siRNA having the general formula 5′-[A]-[B]-[A′]-3′, wherein [A] is a ribonucleotide sequence corresponding to a sequence selected from the group consisting of nucleotides of SEQ ID NOs: 19, 26

[B] is a ribonucleotide sequence consisting of 3 to 23 nucleotides, and

[A′] is a ribonucleotide sequence consisting of the complementary sequence of [A]. The region [A] hybridizes to [A′], and then a loop consisting of region [B] is formed. The loop sequence may be preferably 3 to 23 nucleotide in length. The loop sequence, for example, can be selected from group consisting of following sequences (http://www.ambion.com/techlib/tb/tb_{—}506.html). Furthermore, loop sequence consisting of 23 nucleotides also provides active siRNA (Jacque, J.-M., Triques, K., and Stevenson, M. (2002) Modulation of HIV-1 replication by RNA interference. Nature 418: 435-438.).

CCC, CCACC or CCACACC: Jacque, J. M, Triques, K., and Stevenson, M (2002) Modulation of HIV-1 replication by RNA interference. Nature, Vol. 418: 435-438.

UUCG: Lee, N. S., Dohjima, T., Bauer, G., Li, H., Li, M.-J., Ehsani, A., Salvaterra, P., and Rossi, J. (2002) Expression of small interfering RNAs targeted against HIV-1 rev transcripts in human cells. Nature Biotechnology 20: 500-505. Fruscoloni, P., Zamboni, M., and Tocchini-Valentini, G. P. (2003) Exonucleolytic degradation of double-stranded RNA by an activity in Xenopus laevis germinal vesicles. Proc. Natl. Acad. Sci. USA 100(4): 1639-1644.

UUCAAGAGA: Dykxhoorn, D. M., Novina, C. D., and Sharp, P. A. (2002) Killing the messenger: Short RNAs that silence gene expression. Nature Reviews Molecular Cell Biology 4: 457-467.

For example, preferable siRNAs having hairpin loop structure of the present invention are shown below. In the following structure, the loop sequence can be selected from group consisting of, CCC, UUCG, CCACC, CCACACC, and UUCAAGAGA. Preferable loop sequence is UUCAAGAGA (“ttcaagaga” in DNA). Exemplary hairpin siRNA suitable for use in the context of the present invention include:
for “MGC47816-siRNA:
guguccgcugacagaacaa-[b]-uuguucugucagcggacac (for target sequence of SEQ ID NO: 19)
for HES6-siRNA:
acuuuuagggacccugcag-[b]-cugcagggucccuaaaagu (for target sequence of SEQ ID NO: 26);

The nucleotide sequence of suitable siRNAs can be designed using a siRNA design computer program available from the Ambion website (http://www.ambion.com/techlib/misc/siRNA_finder.html). The computer program selects nucleotide sequences for siRNA synthesis based on the following protocol.

Selection of siRNA Target Sites:
1. Beginning with the AUG start codon of the object transcript, scan downstream for AA dinucleotide sequences. Record the occurrence of each AA and the 3′ adjacent 19 nucleotides as potential siRNA target sites. Tuschl, et al. recommend against designing siRNA to the 5′ and 3′ untranslated regions (UTRs) and regions near the start codon (within 75 bases) as these may be richer in regulatory protein binding sites. UTR-binding proteins and/or translation initiation complexes may interfere with the binding of the siRNA endonuclease complex.
2. Compare the potential target sites to the human genome database and eliminate from consideration any target sequences with significant homology to other coding sequences. The homology search can be performed using BLAST, which can be found on the NCBI server at: www.ncbi.nlm.nih.gov/BLAST/
3. Select qualifying target sequences for synthesis. At Ambion, preferably several target sequences can be selected along the length of the gene for evaluation.

The regulatory sequences flanking the MGC47816 or HES6 genes can be identical or different, such that their expression can be modulated independently, or in a temporal or spatial manner. siRNAs are transcribed intracellularly by cloning the MGC47816 or HES6 gene template into a vector containing, e.g., a RNA pol III transcription unit from the small nuclear RNA (snRNA) U6 or the human H1 RNA promoter. For introducing the vector into the cell, transfection-enhancing agent can be used. FuGENE (Rochediagnostices), Lipofectamin 2000 (Invitrogen), Oligofectamin (Invitrogen), and Nucleofactor (Wako pure Chemical) are useful as the transfection-enhancing agent.

The siRNA of the present invention inhibits the expression of a polypeptide of the invention and is thereby useful for suppressing the biological activity of the polypeptide of the invention. Also, expression-inhibitors, comprising siRNA of the present invention, are useful in that they can inhibit the biological activity of a polypeptide of the invention. Therefore, a composition comprising an antisense oligonucleotide of the present invention, such as an siRNA, is useful in treating an HCC.

Antibodies

Alternatively, the function of a gene product of a gene over-expressed in HCC (e.g., MGC47816 or HES6) can be inhibited by administering a compound that binds to or otherwise inhibits the function of the gene product. For example, the compound may be an antibody which binds to an over-expressed gene product.

The present invention refers to the use of antibodies, particularly antibodies against a protein encoded by an up-regulated marker gene, or a fragment of such an antibody. As used herein, the term “antibody” refers to an immunoglobulin molecule having a specific structure that interacts (i.e., binds) only with the antigen that was used for synthesizing the antibody (i.e., the up-regulated marker gene product) or with an antigen closely related thereto. In the context of the present invention, an antibody may be a fragment of an antibody or a modified antibody, so long as it binds to the protein encoded by the HCC-associated marker gene. For instance, the antibody fragment may be Fab, F(ab′)₂, Fv, or single chain Fv (scFv), in which Fv fragments from H and L chains are ligated by an appropriate linker (Huston J. S. et al. Proc. Natl. Acad. Sci. U.S.A. 85:5879-5883 (1988)). More specifically, an antibody fragment may be generated by treating an antibody with an enzyme, such as papain or pepsin. Alternatively, a gene encoding the antibody fragment may be constructed, inserted into an expression vector, and expressed in an appropriate host cell (see, for example, Co M. S. et al. J. Immunol. 152:2968-2976 (1994); Better M. and Horwitz A. H. Methods Enzymol. 178:476-496 (1989); Pluckthun A. and Skerra A. Methods Enzymol. 178:497-515 (1989); Lamoyi E. Methods Enzymol. 121:652-663 (1986); Rousseaux J. et al. Methods Enzymol. 121:663-669 (1986); Bird R. E. and Walker B. W. Trends Biotechnol. 9:132-137 (1991)).

An antibody may be modified by conjugation with a variety of molecules, such as polyethylene glycol (PEG). The present invention provides such modified antibodies. The modified antibody can be obtained by chemically modifying an antibody. Such modification methods are conventional in the field.

Alternatively, an antibody may comprise a chimeric antibody having a variable region derived from a nonhuman antibody and a constant region derived from a human antibody, or a humanized antibody having a complementarity determining region (CDR) derived from a nonhuman antibody, a frame work region (FR) and a constant region derived from a human antibody. Such antibodies can be prepared by using known technologies.

Cancer therapies directed at specific molecular alterations that occur in cancer cells have been validated through clinical development and regulatory approval of anti-cancer drugs such as trastuzumab (Herceptin) for the treatment of advanced breast cancer, imatinib methylate (Gleevec) for chronic myeloid leukemia, gefitinib (Iressa) for non-small cell lung cancer (NSCLC), and rituximab (anti-CD20 mAb) for B-cell lymphoma and mantle cell lymphoma (Ciardiello F, Tortora G. A novel approach in the treatment of cancer: targeting the epidermal growth factor receptor. Clin Cancer Res. October 2001; 7(10):2958-70. Review.; Slamon D J, Leyland-Jones B, Shak S, Fuchs H, Paton V, Bajamonde A, Fleming T, Eiermann W, Wolter J, Pegram M, Baselga J, Norton L. Use of chemotherapy plus a monoclonal antibody against HER2 for metastatic breast cancer that overexpresses HER2. N Engl J Med. Mar. 15, 2001; 344(11):783-92.; Rehwald U, Schulz H, Reiser M, Sieber M, Staak J O, Morschhauser F, Driessen C, Rudiger T, Muller-Hermelink K, Diehl V, Engert A. Treatment of relapsed CD20+ Hodgkin lymphoma with the monoclonal antibody rituximab is effective and well tolerated: results of a phase 2 trial of the German Hodgkin Lymphoma Study Group. Blood. Jan. 15, 2003; 101(2):420-424.; Fang G, Kim C N, Perkins C L, Ramadevi N, Winton E, Wittmann S and Bhalla K N. (2000). Blood, 96, 2246-2253.). These drugs are clinically effective and better tolerated than traditional anti-cancer agents because they target only transformed cells. Hence, such drugs not only improve survival and quality of life for cancer patients, but also validate the concept of molecularly targeted cancer therapy. Furthermore, targeted drugs can enhance the efficacy of standard chemotherapy when used in combination with it (Gianni L. (2002). Oncology, 63 Suppl 1, 47-56.; Klejman A, Rushen L, Morrione A, Slupianek A and Skorski T. (2002). Oncogene, 21, 5868-5876.). Therefore, future cancer treatments will probably involve combining conventional drugs with target-specific agents aimed at different characteristics of tumor cells such as angiogenesis and invasiveness.

These modulatory methods can be performed ex vivo or in vitro (e.g., by culturing the cell with the agent) or, alternatively, in vivo (e.g., by administering the agent to a subject). The methods involve administering a protein or combination of proteins or a nucleic acid molecule or combination of nucleic acid molecules as therapy to counteract aberrant expression of the differentially expressed genes or the aberrant activity of their gene products.

Diseases and disorders that are characterized by increased (relative to a subject not suffering from the disease or disorder) expression levels or biological activities of the genes or gene products, respectively, may be treated with therapeutics that antagonize (i.e., reduce or inhibit) activity of the over-expressed gene or genes. Therapeutics that antagonize activity can be administered therapeutically or prophylactically.

Accordingly, therapeutics that may be utilized in the context of the present invention include, e.g., (i) antibodies to the MGC47816 or HES6 proteins; (ii) antisense nucleic acids or nucleic acids that are “dysfunctional” (i.e., due to a heterologous insertion within the coding sequence of the MGC47816 or HES6 gene sequence); (iii) small interfering RNA (siRNA); or (iv) modulators (i.e., inhibitors or antagonists that alter the interaction between an MGC47816 or HES6 polypeptide and its binding partner). The dysfunctional antisense molecule is utilized to “knockout” endogenous function of a polypeptide by homologous recombination (see, e.g., Capecchi, Science 244: 1288-1292 1989).

Increased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of a gene whose expression is altered). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

Prophylactic administration occurs prior to the manifestation of overt clinical symptoms of disease, such that a disease or disorder is prevented or, alternatively, delayed in its progression.

Therapeutic methods of the present invention may include the step of contacting a cell with an agent that modulates one or more of the activities of a gene product of a gene differentially expressed in HCC (e.g., MGC47816 or HES6). Examples of agents that modulate protein activity include, but are not limited to, a nucleic acids, proteins, naturally-occurring cognate ligands of such proteins, peptides, peptidomimetics, and other small molecules.

Vaccinating Against HCC

The present invention also relates to a method of treating or preventing HCC in a subject comprising the step of administering to said subject a vaccine comprising a polypeptide encoded by MGC47816 or HES6, an immunologically active fragment of said polypeptide, or a polynucleotide encoding the polypeptide or the fragment thereof. Administration of the polypeptide should induce anti-tumor immunity in a subject. To induce anti-tumor immunity, a polypeptide encoded by MGC47816 or HES6, an immunologically active fragment of said polypeptide, or a polynucleotide encoding such a polypeptide or fragment thereof is administered to subject in need thereof. The polypeptide or the immunologically active fragments thereof are useful as vaccines against HCC. In some cases, the proteins or fragments thereof may be administered in a form bound to the T cell receptor (TCR) or presented by an antigen presenting cell (APC), such as macrophage, dendritic cell (DC), or B-cells. Due to the strong antigen presenting ability of DC, the use of DC is most preferable among the APCs.

In the context of the present invention, a vaccine against HCC refers to a substance that has the ability to induce anti-tumor immunity upon inoculation into animals. According to the present invention, polypeptides encoded by MGC47816 or HES6, or fragments thereof, were suggested to be HLA-A24 or HLA-A*0201 restricted epitope peptides that may induce potent and specific immune response against HCC cells expressing MGC47816 or HES6. Thus, the present invention also encompasses method of inducing anti-tumor immunity using such polypeptides. In general, anti-tumor immunity includes immune responses such as follows:
induction of cytotoxic lymphocytes against tumors,
induction of antibodies that recognize tumors, and
induction of anti-tumor cytokine production.

Therefore, when a certain protein induces any one of these immune responses upon inoculation into an animal, the protein is determined to have anti-tumor immunity inducing effect. The induction of the anti-tumor immunity by a protein can be detected by observing in vivo or in vitro the response of the immune system in the host against the protein.

For example, a method for detecting the induction of cytotoxic T lymphocytes is well known. Specifically, a foreign substance that enters the living body is presented to T cells and B cells by the action of antigen presenting cells (APCs). T cells that respond in an antigen specific manner to the antigen presented by the APCs differentiate into cytotoxic T cells (or cytotoxic T lymphocytes; CTLs) due to stimulation by the antigen, and then proliferate (this is referred to as activation of T cells). Therefore, CTL induction by a certain peptide can be evaluated by presenting the peptide to a T cell via an APC, and then detecting the induction of CTLs. Furthermore, APCs have the effect of activating CD4+ T cells, CD8+ T cells, macrophages, eosinophils, and NK cells. Since CD4+ T cells and CD8+ T cells are also important in anti-tumor immunity, the anti-tumor immunity-inducing action of the peptide can be evaluated using the activation effect of these cells as indicators.

A method for evaluating the inducing action of CTLs using dendritic cells (DCs) as the APC is well known in the art. DCs are representative APCs having the strongest CTL-inducing action among APCs. In this method, the test polypeptide is initially contacted with a DC, and then this DC is contacted with T cells. Detection of T cells having cytotoxic effects against the cells of interest after the contact with DC shows that the test polypeptide has an activity of inducing the cytotoxic T cells. Activity of CTLs against tumors can be detected, for example, using the lysis of ⁵¹Cr-labeled tumor cells as the indicator. Alternatively, the method of evaluating the degree of tumor cell damage using ³H-thymidine uptake activity or LDH (lactose dehydrogenase)-release as the indicator is also well known.

Apart from DCs, peripheral blood mononuclear cells (PBMCs) may also be used as APCs. The induction of CTL has been reported to be enhanced by culturing PBMCs in the presence of GM-CSF and IL-4. Similarly, CTLs have been shown to be induced by culturing PBMCs in the presence of keyhole limpet hemocyanin (KLH) and IL-7.

Test polypeptides confirmed to possess CTL-inducing activity by these methods are deemed to be polypeptides having DC activation effect and subsequent CTL-inducing activity. Therefore, polypeptides that induce CTLs against tumor cells are useful as vaccines against tumors. Furthermore, APC that have acquired the ability to induce CTLs against tumors through contact with the polypeptides are also useful as vaccines against tumors. Furthermore, CTLs that have acquired cytotoxicity due to presentation of the polypeptide antigens by APCs can be also used as vaccines against tumors. Such therapeutic methods for tumors using anti-tumor immunity due to APCs and CTLs are referred to as cellular immunotherapy.

Generally, when using a polypeptide for cellular immunotherapy, efficiency of the CTL-induction is known to be increased by combining a plurality of polypeptides having different structures and contacting them with DCs. Therefore, when stimulating DC with protein fragments, it is advantageous to use a mixture of multiple types of fragments.

Alternatively, the induction of anti-tumor immunity by a polypeptide can be confirmed by observing the induction of antibody production against tumors. For example, when antibodies against a polypeptide are induced in a laboratory animal immunized with the polypeptide, and when growth of tumor cells is suppressed by those antibodies, the polypeptide is deemed to have the ability to induce anti-tumor immunity.

Anti-tumor immunity is induced by administering the vaccine of this invention, and the induction of anti-tumor immunity enables treatment and prevention of HCC. Therapy against cancer or prevention of the onset of cancer includes any of the following steps, such as inhibition of the growth of cancerous cells, involution of cancer, and suppression of the occurrence of cancer. A decrease in mortality or morbidity of individuals having cancer, a decrease in the levels of tumor markers in the blood, alleviation of detectable symptoms accompanying cancer, and such are also included in the therapy or prevention of cancer. Such therapeutic and preventive effects are preferably statistically significant, for example, in observation, at a significance level of 5% or less, wherein the therapeutic or preventive effect of a vaccine against cell proliferative diseases is compared to a control without vaccine administration. For example, Student's t-test, the Mann-Whitney U-test, or ANOVA may be used for statistical analysis.

The above-mentioned protein having immunological activity or a vector encoding the protein may be combined with an adjuvant. An adjuvant refers to a compound that enhances the immune response against the protein when administered together (or successively) with the protein having immunological activity. Exemplary adjuvants include, but are not limited to, cholera toxin, salmonella toxin, alum, and such. Furthermore, the vaccine of this invention may be combined appropriately with a pharmaceutically acceptable carrier. Examples of such carriers are sterilized water, physiological saline, phosphate buffer, culture fluid, and such. Furthermore, the vaccine may contain as necessary, stabilizers, suspensions, preservatives, surfactants, and such. The vaccine can be administered systemically or locally. Vaccine administration can be performed by single administration, or boosted by multiple administrations.

When using an APC or CTL as the vaccine of this invention, tumors can be treated or prevented, for example, by the ex vivo method. More specifically, PBMCs of the subject receiving treatment or prevention are collected, the cells are contacted with the polypeptide ex vivo, and following the induction of APCs or CTLs, the cells may be administered to the subject. APCs can be also induced by introducing a vector encoding the polypeptide into PBMCs ex vivo. APCs or CTLs induced in vitro can be cloned prior to administration. By cloning and growing cells having high activity of damaging target cells, cellular immunotherapy can be performed more effectively. Furthermore, APCs and CTLs isolated in this manner may be used for cellular immunotherapy not only against individuals from whom the cells are derived, but also against similar types of tumors from other individuals.

Furthermore, a pharmaceutical composition for treating or preventing a cell proliferative disease, such as cancer, comprising a pharmaceutically effective amount of the polypeptide of the present invention is provided. The pharmaceutical composition may be used for raising anti tumor immunity.

Pharmaceutical Compositions for Inhibiting HCC

In the context of the present invention, suitable pharmaceutical formulations include those suitable for oral, rectal, nasal, topical (including buccal and sub-lingual), vaginal or parenteral (including intramuscular, sub-cutaneous and intravenous) administration, or for administration by inhalation or insufflation. Preferably, administration is intravenous. The formulations are optionally packaged in discrete dosage units.

Pharmaceutical formulations suitable for oral administration include capsules, cachets or tablets, each containing a predetermined amount of active ingredient. Suitable formulations also include, but are not limited to, powders, granules or solutions, suspensions and emulsions. The active ingredient is optionally administered as a bolus electuary or paste. Tablets and capsules for oral administration may contain conventional excipients such as binding agents, fillers, lubricants, disintegrant and/or wetting agents. A tablet may be made by compression or molding, optionally with one or more formulational ingredients. Compressed tablets may be prepared by compressing in a suitable machine the active ingredients in a free-flowing form such as a powder or granules, optionally mixed with a binder, lubricant, inert diluent, lubricating, surface active and/or dispersing agent. Molded tablets may be made by molding in a suitable machine a mixture of the powdered compound moistened with an inert liquid diluent. The tablets may be coated according to methods well known in the art. Oral fluid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), and/or preservatives. The tablets may optionally be formulated so as to provide slow or controlled release of the active ingredient therein. A package of tablets may contain one tablet to be taken on each of the month.

Formulations for suitable parenteral administration include aqueous and non-aqueous sterile injection solutions, optionally containing anti-oxidants, buffers, bacteriostats and solutes which render the formulation isotonic with the blood of the intended recipient; as well as aqueous and non-aqueous sterile suspensions, optionally including suspending agents and/or thickening agents. The formulations may be presented in unit dose or multi-dose containers, for example as sealed ampoules and vials, and may be stored in a freeze-dried (lyophilized) condition, requiring only the addition of the sterile liquid carrier, for example, saline, water-for-injection, immediately prior to use. Alternatively, the formulations may be presented for continuous infusion. Extemporaneous injection solutions and suspensions may be prepared from sterile powders, granules and tablets of the kind previously described.

Formulations for suitable rectal administration include suppositories with standard carriers such as cocoa butter or polyethylene glycol. Formulations for suitable topical administration in the mouth, for example buccally or sublingually, include lozenges, containing the active ingredient in a flavored base such as sucrose and acacia or tragacanth, and pastilles comprising the active ingredient in a base such as gelatin and glycerin or sucrose and acacia. For intra-nasal administration, the compounds of the invention may be used as a liquid spray, a dispersible powder or in the form of drops. Drops may be formulated with an aqueous or non-aqueous base also comprising one or more dispersing agents, solubilizing agents and/or suspending agents.

For administration by inhalation, the compounds can be conveniently delivered from an insufflator, nebulizer, pressurized packs or other convenient means of delivering an aerosol spray. Pressurized packs may comprise a suitable propellant such as dichlorodifluoromethane, trichlorofluoromethane, dichiorotetrafluoroethane, carbon dioxide or other suitable gas. In the case of a pressurized aerosol, the dosage unit may be determined by providing a valve to deliver a metered amount.

Alternatively, for administration by inhalation or insufflation, the compounds may take the form of a dry powder composition, for example a powder mix of the compound and a suitable powder base, such as lactose or starch. The powder composition may be presented in unit dosage form, for example, as capsules, cartridges, gelatin or blister packs from which the powder may be administered with the aid of an inhalator or insufflators.

Other formulations include implantable devices and adhesive patches; which release a therapeutic agent.

When desired, the above described formulations, adapted to give sustained release of the active ingredient, may be employed. The pharmaceutical compositions may also contain other active ingredients such as antimicrobial agents, immunosuppressants and/or preservatives.

It should be understood that in addition to the ingredients particularly mentioned above, the formulations of this invention may include other agents conventional in the art with regard to the type of formulation in question. For example, formulations suitable for oral administration may include flavoring agents.

Preferred unit dosage formulations contain an effective dose, as recited below, or an appropriate fraction thereof, of the active ingredient.

For each of the aforementioned conditions, the compositions, e.g., polypeptides and organic compounds, can be administered orally or via injection at a dose ranging from about 0.1 to about 250 mg/kg per day. The dose range for adult humans is generally from about 5 mg to about 17.5 g/day, preferably about 5 mg to about 10 g/day, and most preferably about 100 mg to about 3 g/day. Tablets or other unit dosage forms of presentation provided in discrete units may conveniently contain an amount which is effective at such dosage or as a multiple of the same, for instance, units containing about 5 mg to about 500 mg, usually from about 100 mg to about 500 mg.

The dose employed will depend upon a number of factors, including the age and sex of the subject, the precise disorder being treated, and its severity. Also the route of administration may vary depending upon the condition and its severity. In any event, appropriate and optimum dosages may be routinely calculated by those skilled in the art, taking into consideration the above-mentioned factors.

Aspects of the present invention are further described in the following examples. These examples are illustrative only and are not intended to limit the scope of the invention described in the claims.

### EXAMPLE 1

### Materials and General Methods

Patients and Tissue Specimens

All hepatocellular carcinoma tissues and the corresponding non-cancerous tissues were obtained with informed consent from surgical specimens of patients who underwent surgery.

Genome-Wide cDNA Microarray

In this study, a genome-wide cDNA microarray with 23,040 genes was used. Total RNA extracted from the microdissected tissue was treated with DNase I, amplified with Ampliscribe T7 Transcription Kit (Epicentre Technologies), and subsequently labeled during reverse transcription with a Cy-dye (Amersham); RNA from non-cancerous tissue was labeled with Cy5 and RNA from tumor with Cy3. Hybridization, washing, and detection were carried out as described previously (Ono, K., et al. Cancer Res., 60: 5007-5011, (2000)), and fluorescence intensity of Cy5 and Cy3 for each target spot was generated by ArrayVision software (Amersham Pharmacia). After subtraction of background signal, the duplicate values were averaged for each spot. Then, all fluorescence intensities on a slide were normalized to adjust the mean Cy5 and Cy3 intensities of 52 housekeeping genes for each slide. Genes were excluded from further investigation when the intensities of both Cy3 and Cy5 were below 25,000 fluorescence units, and of the remainder, those with Cy3/Cy5 signal ratios >2.0 were selected for further evaluation.

Cell Lines

Human hepatoma cell lines Alexander and HepG2 and monkey fibroblast cell line COS7 were obtained from the American Type Culture Collection (ATCC). Another human hepatoma cell line Huh7 was obtained from Japanese Collection of Research Bioresources (JCRB), while SNU423, SNU449 and SNU475 were obtained from the Korea cell-line bank. All cell lines were grown in monolayers in appropriate media: Dulbecco's modified Eagle's medium for Alexander, Huh7, HepG2 and COS7; RPMI1640 for SNU423, SNU449 and SNU475 supplemented with 10% fetal bovine serum and 1% antibiotic/antimycotic solution (Sigma). All cells were maintained at 37° C. in humid air with 5% CO₂, (Alexander, Huh7, HepG2, SNU423, SNU449, SNU475, and COS7).

RNA Preparation and RT-PCR

Total RNA was extracted with a Qiagen RNeasy kit (Qiagen) or Trizol reagent (Life Technologies, Inc.) according to the manufacturers' protocols. Ten-microgram aliquots of total RNA were reversely transcribed for single-stranded cDNAs using poly dT₁₂₋₁₈ primer (Amersham Pharmacia Biotech) with Superscript II reverse transcriptase (Life Technologies). Each single-stranded cDNA preparation was diluted for subsequent PCR amplification by standard RT-PCR experiments carried out in 12 μl volumes of PCR buffer (TAKARA). Amplification proceeded for 4 min at 94° C. for denaturing, followed by 21 (for GAPDH), 35 (for MGC47816) cycles of 94° C. for 30 s, 60° C. for 30 s, and 72° C. for 60 s, and 35 (for HES6) cycles of 94° C. for 30 s, 60° C. for 40 s, and 72° C. for 60 s, in the GeneAmp PCR system 9700 (Perkin-Elmer, Foster City, Calif.). Primer sequences were as follows:

| | |
|---|---|
| for GAPDH: forward, | |
| 5′-ACAACAGCCTCAAGATCATCAG-3′ | (SEQ ID NO: 3) |
| and | |
| reverse, | |
| 5′-GGTCCACCACTGACACGTTG-3′; | (SEQ ID NO: 4) |
| for MGC47816: forward, | |
| 5′-CAAATAGGCAGACTGGAAAGATG-3′ | (SEQ ID NO: 5) |
| and | |
| reverse: | |
| 5′-CTAGGGAAGCAGTAGGATTTGGT-3′; | (SEQ ID NO: 6) |
| for HES6: forward, | |
| 5′-GAGCTCCTGAACCATCTGCTC-3′ | (SEQ ID NO: 20) |
| and | |
| reverse: | |
| 5′-CAAGATGTACAGAGCATCACAGC-3′; | (SEQ ID NO: 21) |

Northern-Blot Analysis

Human multiple-tissue blots (Clontech, Palo Alto, Calif.) were hybridized with α ³²P-labeled PCR product of MGC47816 and HES6. Pre-hybridization, hybridization and washing were performed according to the supplier's recommendations. The blots were autoradiographed with intensifying screens at −80° C. for 72 h.

Construction of Expression Vector

The entire coding region of MGC47816 was amplified by RT-PCR using the following gene specific sets of primers: 5′-ATTGTCGACGCTCGCCCTACTGAGCGAGCG-3′ (SEQ ID NO: 7), and 5′-AATCTCGAGAGCAGGAATTCACTTAAGTTTTAACTC-3′ (SEQ ID NO: 8).

The entire coding region of HES6 was amplified by RT-PCR using the following gene-specific set of primers: 5′-ATTGAATTCGCATGGCGCCACCCGCGGCG-3′ (SEQ ID NO: 22), and 5′-AATGGTACCTCACCAAGGCCTCCAGACACTCC-3′ (SEQ ID NO: 23). The PCR product was cloned into an appropriate cloning site of pCMV-HA vector (CLONTECH).

Immunoblotting

Cells transfected with pCMV-HA-MGC47816 and pCMV-HA-HES6 were washed twice with PBS and harvested in lysis buffer (150 mM NaCl, 1% Triton X-100, 50 mM Tris-HCl pH 7.4, 1 mM DTT, and 1× complete Protease Inhibitor Cocktail (Boehringer)). After the cells were homogenized and centrifuged at 10,000×g for 30 min, the supernatant was standardized for protein concentration by the Bradford assay (Bio-Rad). Proteins were separated by 10% SDS-PAGE and immunoblotted with rat anti-HA (Roche) antibody. HRP-conjugated goat anti-rat IgG (Santa Cruz) served as the secondary antibody for the ECL Detection System (Amersham).

Immunohistochemical Staining

Cells transfected with pCMV-HA-MGC47816 and pCMV-HA-HES6 were fixed with PBS containing 4% paraformaldehyde for 15 min, then rendered permeable with PBS containing 0.1% Triton X-100 for 2.5 min at RT. Subsequently, the cells were covered with 2% BSA in PBS for 12 h at 4° C. to block non-specific hybridization. Rat anti-HA (ROCHE) antibody at 1:1000 dilution was used for the first antibody, and the reaction was visualized after incubation with RHODAMINE-conjugated anti-rat second antibody (Leinco and ICN). Nuclei were counter-stained with 4′,6′-diamidine-2′-phenylindole dihydrochloride (DAPI). Fluorescent images were obtained under an ECLIPSE E800 microscope.

Construction and Effect of Plasmids Expressing MGC47816-siRNA and HES6-siRNA

To prepare plasmid vector expressing short interfering RNA (siRNA), the genomic fragment of the H1RNA gene containing its promoter region was amplified by PCR, using the following set of primers, 5′-TGGTAGCCAAGTGCAGGTTATA-3′ (SEQ ID NO: 9) and 5′ -CCAAAGGGTTTCTGCAGTTTCA-3′ (SEQ ID NO: 10) for H1RNA, and human placental DNA as a template. The products were purified and cloned into pCR2.0 plasmid vector using a TA cloning kit according to the supplier's protocol (Invitrogen). The BamHI and XhoI fragment containing H1RNA was cloned into nucleotides 1257 to 56 fragment of pcDNA3.1(+) plasmid, which was amplified by PCR using 5′-TGCGGATCCAGAGCAGATTGTACTGAGAGT-3′ (SEQ ID NO: 11) and 5′-CTCTATCTCGAGTGAGGCGGAAAGAACCA-3′ (SEQ ID NO: 12). The ligated DNA became the template for PCR amplification with primers, 5′-TTTAAGCTTGAAGACCATTTTTGGAAAAAAAAAAAAAAAAAAAAAAC-3′ (SEQ ID NO: 13) and 5′-TTTAAGCTTGAAGACATGGGAAAGAGTGGTCTCA-3′ (SEQ ID NO: 14) for H1RNA. The product was digested with HindIII, and subsequently self-ligated to produce psiH1BX3.0 vector plasmid. Control plasmid, psiH1BX-EGFP was prepared by cloning double-stranded oligonucleotides of 5′-CACCGAAGCAGCACGACTTCTTCTTCAAGAGAGAAGAAGTCGTGCTGCTTC-3′ (SEQ ID NO: 15) and 5′-AAAAGAAGCAGCACGACTTCTTCTCTCTTGAAGAAGAAGTCGTGCTGCTTC-3′ (SEQ ID NO: 16) into the BbsI site in the psiH1BX3.0 vector. Plasmids expressing MGC47816-siRNAs and HES6-siRNAs were prepared by cloning of double-stranded oligonucleotides into psiH1BX3.0 vector. The oligonucleotides used for MGC47816-siRNAs were

| | |
|---|---|
| 5′-TCCCGTGTCCGCTGACAGAACAATTCAAGAG | (SEQ ID NO: 17) |
| ATTGTTCTGTCAGCGGACAC-3′ | |
| and | |
| 5′-AAAAGTGTCCGCTGACAGAACAATCTCTTGA | (SEQ ID NO: 18) |
| ATTGTTCTGTCAGCGGACAC-3′ | |
| (psiH1BX-MGC47816-3). | |

The oligonucleotides used for HES6-siRNAs were

| | |
|---|---|
| 5′-TCCCACTTTTAGGGACCCTGCAGTTCAAGAG | (SEQ ID NO: 24) |
| ACTGCAGGGTCCCTAAAAGT-3′ | |
| and | |
| 5′-AAAAACTTTTAGGGACCCTGCAGTCTCTTGA | (SEQ ID NO: 25) |
| ACTGCAGGGTCCCTAAAAGT-3′ | |
| (psiH1BX-HES6-2). | |

Plasmids, psiH1BX-MGC47816-3, were transfected into Alexander and SNU449 cells, psiH1BX-HES6-2 were transfected into Alexander and HepG2 cells using FuGENE6 reagent (Roche) or Nucleofector reagent (Alexa) according to the supplier's recommendations. Total RNA was extracted from the cells 48 hours after the transfection. Cells were cultured in the presence of 400-800 μg/ml geneticin (G418) for 14 days and stained with Giemsa's solution (MERCK, Germany) as described elsewhere.

MTT Assay

Cells (1×10⁶) on 10 cm-dish were transfected with a siRNA expression vector or control vector using FuGene6 (Roche) according to the supplier's protocol. Cell viability was evaluated by MTT assay seven days after transfection. Cell-counting kit-8 (DOJINDO) was added to each dish at a concentration of 1/10 volume, and the plates were incubated at 37° C. for an additional 2 h; then absorbance was measured at 490 nm, and at 630 nm as reference, with a Microplate Reader 550 (Bio-Rad Laboratories, Hercules, Calif.).

Statistical Analysis

The data were subjected to analysis of variance (ANOVA) and the Scheffé's F test.

### EXAMPLE 2

### Results

Identification of D4999 Whose expression is Frequently Up-Regulated in Human HCC

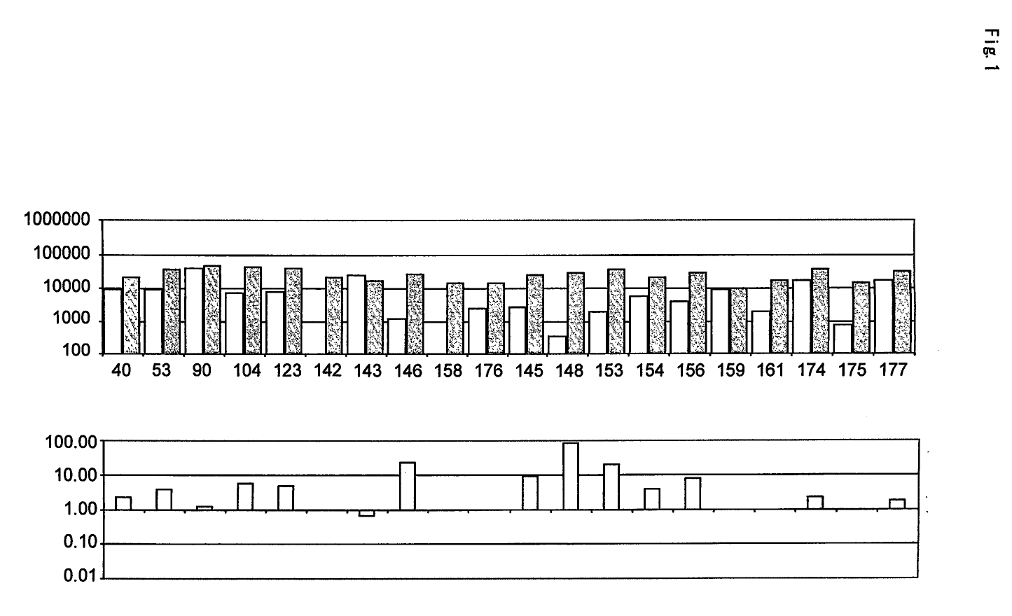

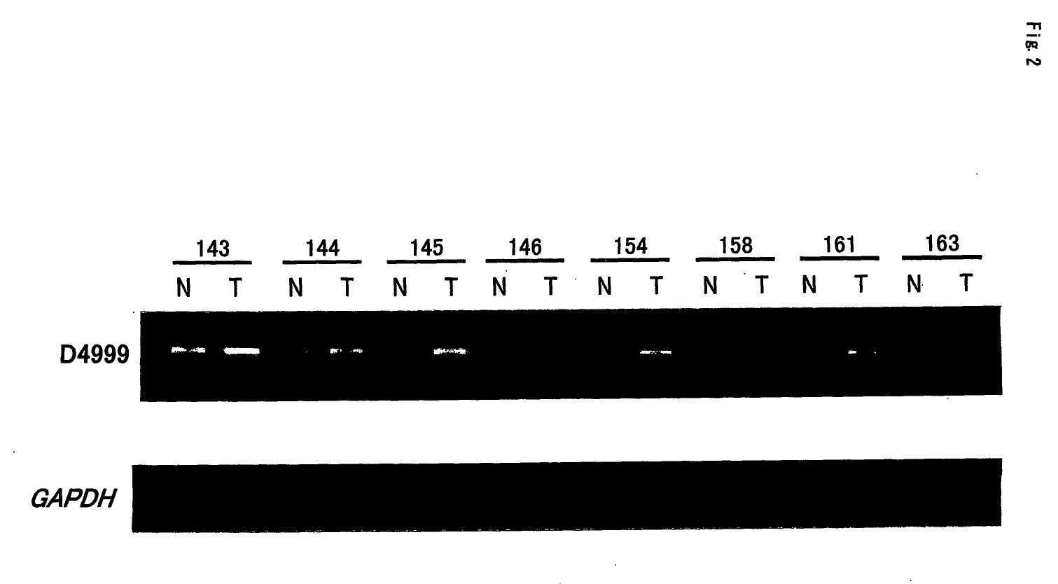

The expression profiles of 20 HCCs were compared with their corresponding non-cancerous liver tissues using cDNA microarray containing 23,040 genes. Among commonly up-regulated genes in HCCs, a gene with an in-house accession number of D4999, corresponding to an EST (MGC47816) in Hs.420244 of a UniGene cluster (http://www.ncbi.nlm.nih.gov/UniGene/), was over-expressed in seven of eleven HCCs compared with the corresponding noncancerous liver tissues (FIG. 1). To clarify the results of the microarray, semi-quantitative RT-PCR we performed, which revealed that expression of D4999 was increased in seven of additional eight HCCs as compared with corresponding non-cancerous liver tissues (FIG. 2).

Identification, Expression, and Structure of MGC47816

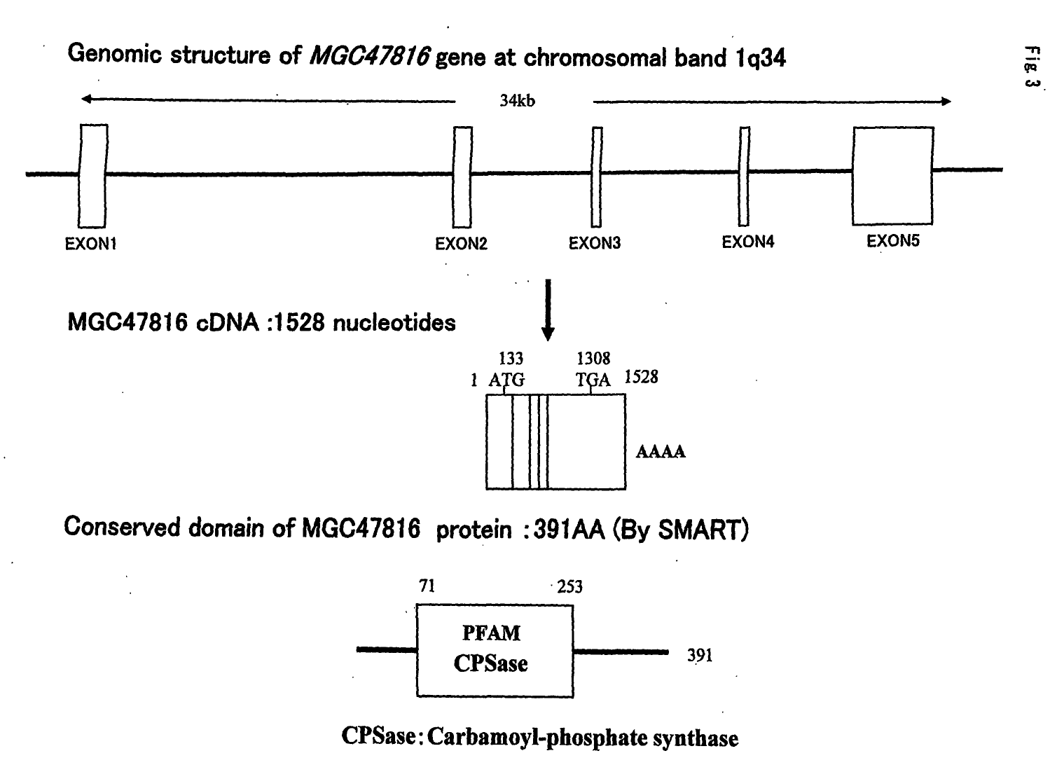

Homology searches with the sequence of D4999 in public databases using BLAST program in National Center for Biotechnology Information (http://www.ncbi.nlm.nih.gov/BLAST/) identified ESTs including MGC47816 (GenBank accession number of NM_{—}173642) and a genomic sequence with GenBank accession number of AA971400 assigned to chromosomal band 1q34.1. Comparison of MGC47816 cDNA and the genomic sequence disclosed that this gene consisted of 5 exons. The putative full-length cDNA consisted of 1528 nucleotides, with an open reading frame of 1176 nucleotides (SEQ ID NO: 1) encoding a 391-amino-acid protein (SEQ ID NO: 2). The amino acid sequence of the predicted MGC47816 protein showed 88% identity to a mouse hypothetical protein B930030J24. A search for protein motifs with the Simple Modular Architecture Research Tool (SMART, http://smart.embl-heidelberg.de) revealed that the predicted protein contained a carbamoyl-phosphate synthase L chain and an ATP binding domain (codons 71-253) (FIG. 3).

Subcellular Localization of HA-Tagged MGC47816 Protein

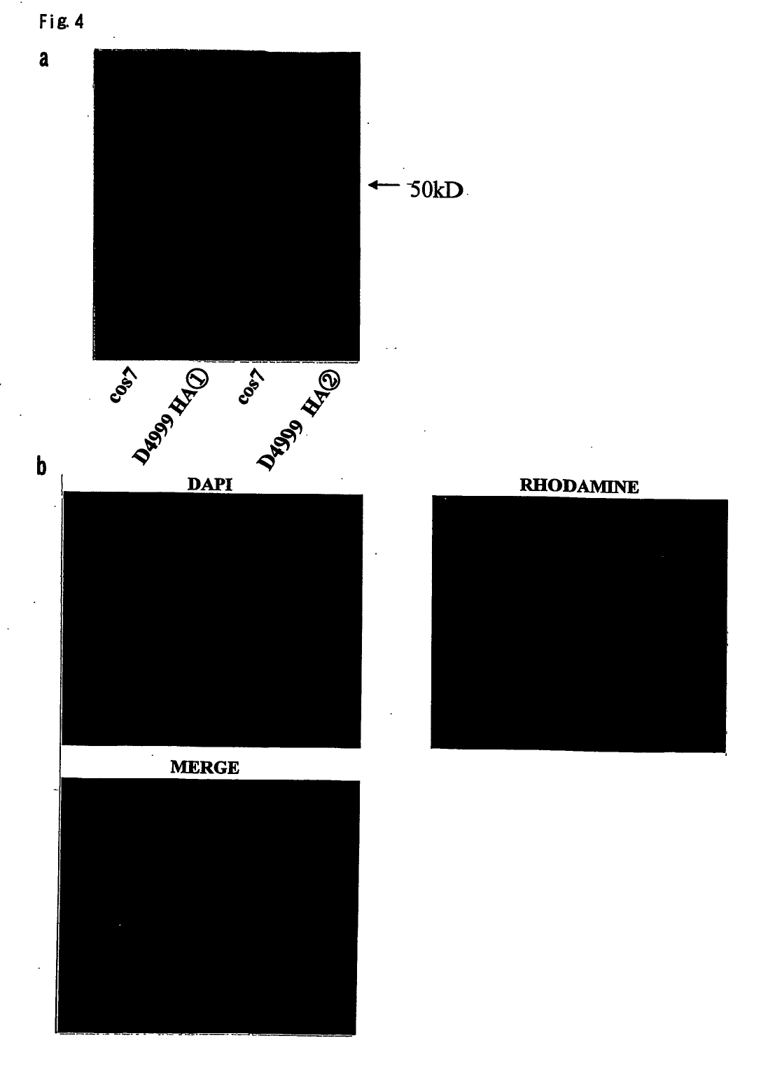

To investigate the subcellular localization of the MGC47816 protein, a plasmid expressing HA-tagged (pCMV-HA-MGC47816) were transiently transfected into COS7 cells. Western blot analysis using extracts from the cells and anti-HA antibody revealed a 50-KDa band corresponding to the tagged protein (FIG. 4a). Subsequent immunohistochemical staining of the cells with these antibodies indicated that the protein was mainly present in the cytoplasm (FIG. 4b).

Effect of Plasmids Expressing MGC47816-siRNA on Growth of HCC Cells

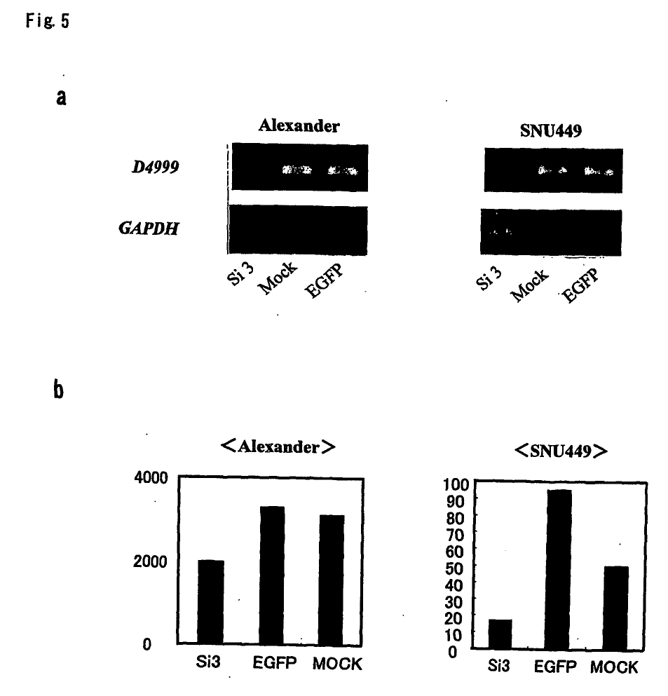

To investigate the function of the MGC47816 protein in cancer cells, plasmids expressing MGC47816-siRNA were constructed and their effect on MGC47816 expression was examined. Transfection of Alexander and SNU449 cells with psiH1BX-MGC47816-3 (Si-3), psiH1BX-EGFP (EGFP) or psiH1BX-mock (Mock) revealed that psiH1BX-MGC47816-3 (Si-3) significantly suppressed expression of MGC47816 in the cells compared to psiH1BX-EGFP (EGFP) or psiH1BX-mock (Mock) (FIG. 5a). To test whether the suppression of MGC47816 results in growth suppression of hepatocellular carcinoma cells, Alexander and SNU449 cells were transfected with psiH1BX-MGC47816-3 (Si-3), psiH1BX-EGFP (EGFP) or psiH1BX-mock (Mock). Viable cells transfected with psiH1BX-MGC47816-3 (Si-3) were markedly reduced compared to those transfected with psiH1BX-EGFP (EGFP) or psiH1BX-mock (Mock) suggesting that decreased expression of MGC47816 suppressed growth of hepatocellular carcinoma cells (FIG. 5b).

Identification of C2298 Whose Expression was Frequently Elevated in HCCs

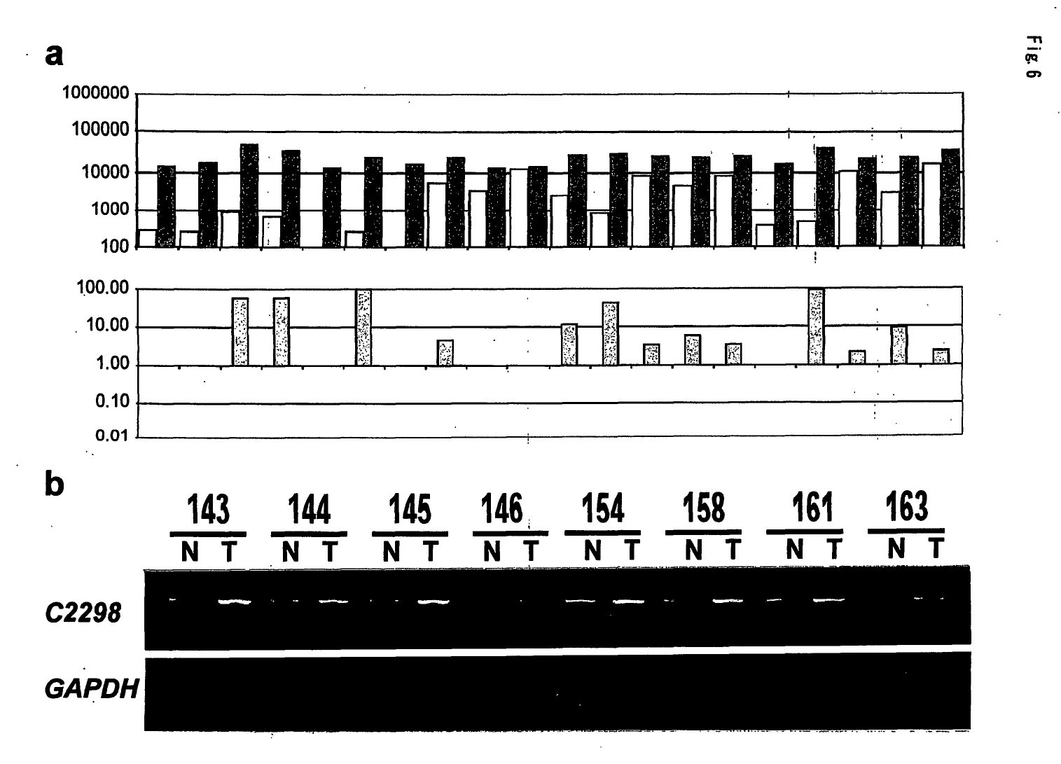

The expression profiles of 20 HCCs were analyzed with the corresponding non-cancerous liver tissues using the cDNA microarray containing 23,040 genes. Among commonly up-regulated genes in HCCs, a gene with an in-house accession number of C2298, corresponding to HES6 (Hs.42949 of a UniGene cluster at http://www.ncbi.nlm.nih.gov/UniGene/), was over-expressed in eleven of twelve HCCs compared with the corresponding noncancerous liver tissues (FIG. 6a). To clarify the results of the microarray, semi-quantitative RT-PCR was performed, which revealed that expression of HES6 was increased in 7 out of additional 8 HCCs as compared with corresponding non-cancerous liver tissues (FIG. 6b).

Identification, Expression, and Structure of HES6

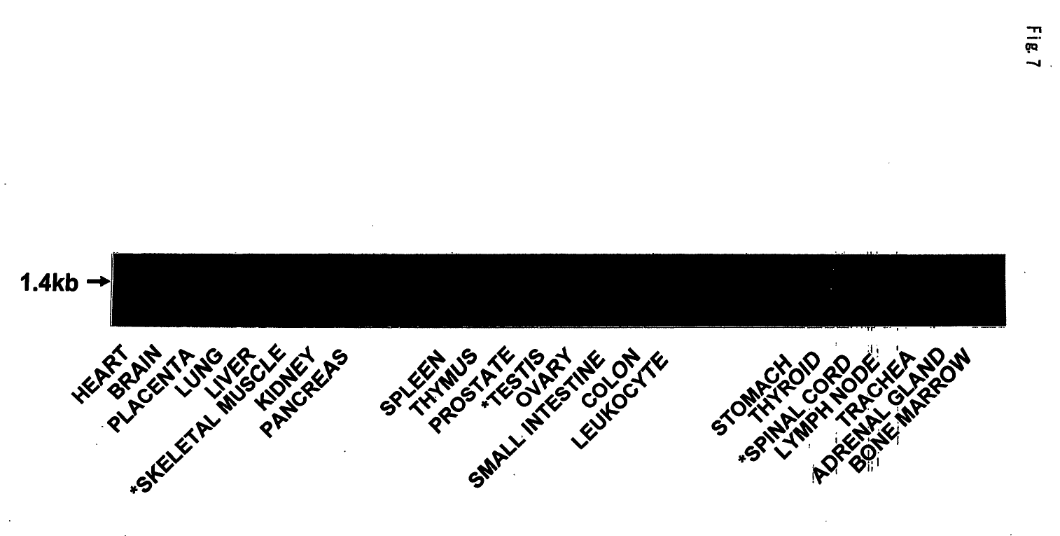

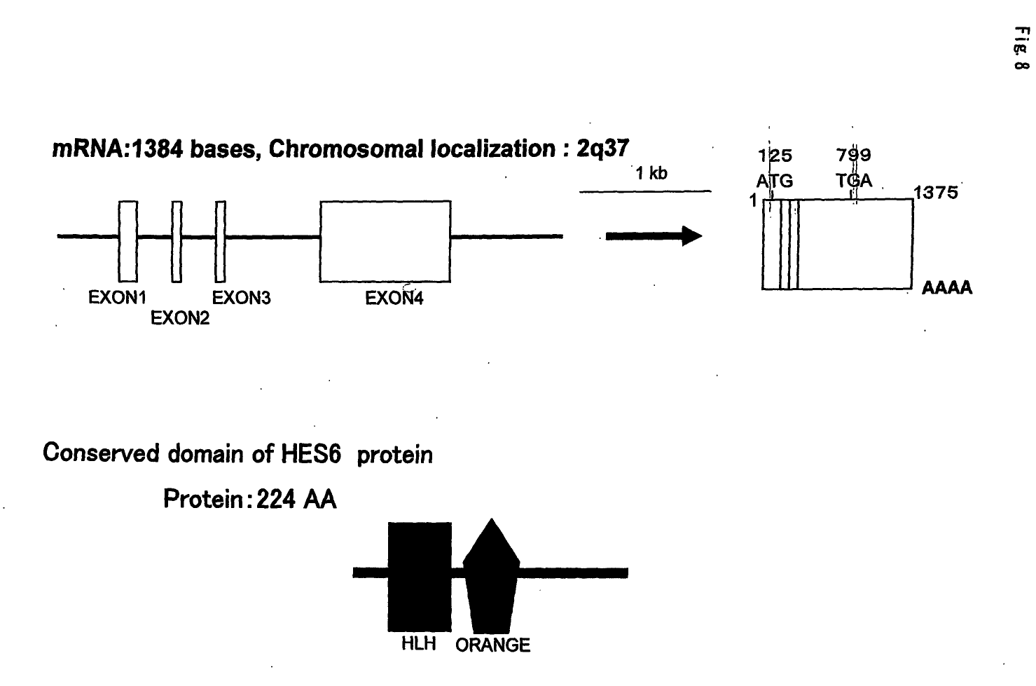

Homology searches with the sequence of C2298 in public databases using BLAST program in National Center for Biotechnology Information (http://www.ncbi.nlm.nih.gov/BLAST/) identified cDNA sequences including GenBank accession number BC007939 that corresponded to HES6, and a genomic sequence with GenBank accession number of AA357675 assigned to chromosomal band 2q37. The HES6 cDNA sequence consisted of 1375 nucleotides containing an open reading frame of 675 nucleotides (SEQ ID NO: 27) encoding a putative 224-amino-acid protein(SEQ ID NO: 28) (GenBank accession number BC007939). The first ATG was flanked by a sequence (GGCATGG) that agreed with the consensus sequence for initiation of translation in eukaryotes. Comparison of HES6 cDNA and the genomic sequence disclosed that this gene consisted of 4 exons. Additionally, multiple-Tissue Northern-blot analysis was performed using a PCR product of HES6 as a probe, and detected a 1.4 kb-transcript that was expressed in testis, spinal code and skeletal muscle (FIG. 7). A search for protein motifs with the Simple Modular Architecture Research Tool (SMART, http://smart.emblheidelberg.de) revealed that the predicted protein contained a helix-loop-helix domain and orange domain (codons 31-80, 94-135) (FIG. 8).

Subcellular Localization of HA-Tagged HES6 Protein

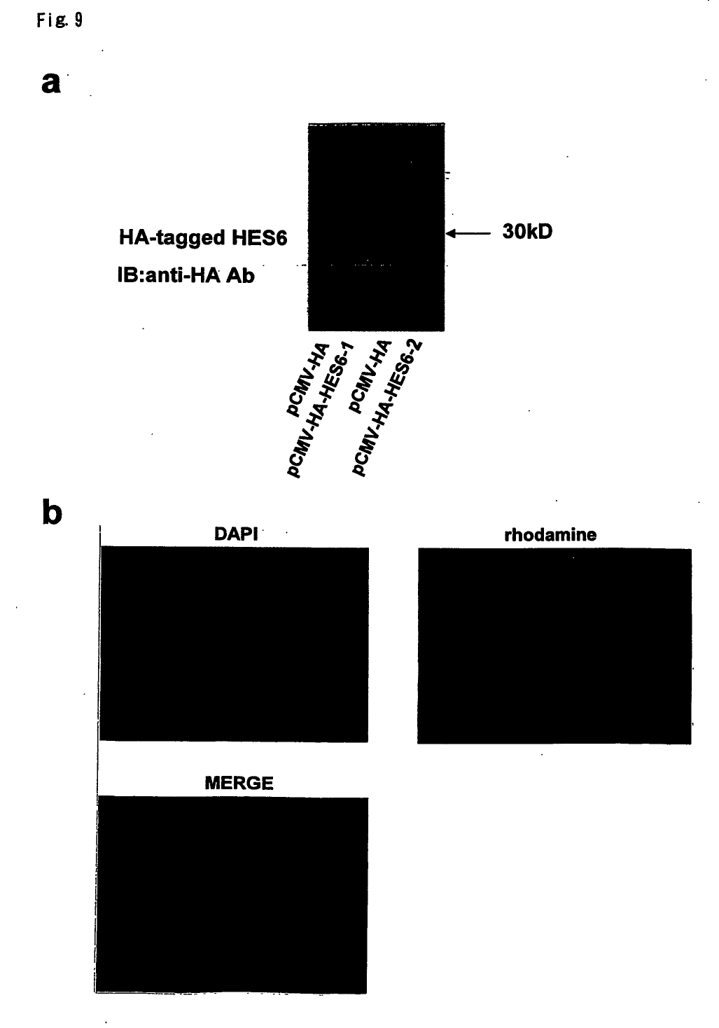

To investigate the subcellular localization of the HES6 protein, a plasmid expressing HA-tagged (pCMV-HA-HES6) was transiently transfected into COS7 cells. Western blot analysis using extracts from the cells and anti-HA antibody revealed a 30-Da band corresponding to the tagged protein (FIG. 9a). Subsequent immunohistochemical staining of the cells with these antibodies indicated that the protein was mainly present in the nucleus (FIG. 9b).

Effect of Plasmids Expressing HES6-siRNA on Growth of Hepatocellular Carcinoma Cells.

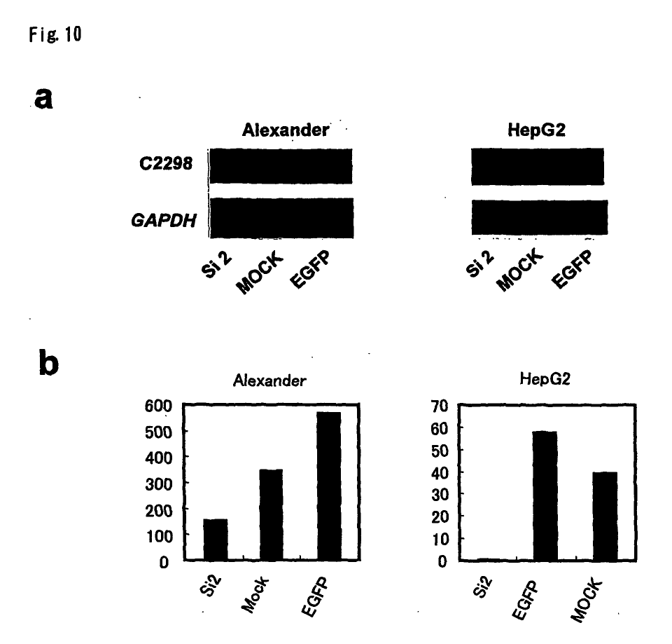

To investigate the function of HES6 in cancer cells, plasmids expressing HES6-siRNA were constructed and their effect on HES6 expression was examined. Transfection of Alexander and HepG2 cells with psiH1BX-HES6-2, psiH1BX-EGFP or psiH1BX-mock revealed that psiH1BX-HES6-2 significantly suppressed expression of HES6 in the cells compared to psiH1BX-EGFP or psiH1BX-mock (FIG. 10a). To test whether the suppression of HES6 results in growth suppression of HCC cells, Alexander and HepG2 cells were transfected with psiH1BX-HES6-2, psiH1BX-EGFP or psiH1BX-mock. Viable cells transfected with psiH1BX-HES6-2 were markedly reduced compared to those transfected with psiH1BX-EGFP or psiH1BX-mock suggesting that decreased expression of HES6 suppressed growth of hepatocellular carcinoma cells (FIG. 10b).

### EXAMPLE 6

### Disucussion

cDNA microarray technologies have enabled the discovery of comprehensive profiles of gene expression in various human neoplasms. This approach discloses the complex nature of cancer cells, and enables a more profound understanding of carcinogenesis. In addition, it facilitates the identification of genes whose expression levels are deregulated in tumors, which should lead to more precise diagnosis of the tumors, and the development of novel therapeutic strategies.

Studies designed to reveal mechanisms of carcinogenesis have identified several molecular targets for anti-tumor agents. For example, inhibitors of farnesyltransferase (FTIs) were originally developed to inhibit the growth-signaling pathway related to Ras, whose activation depends upon posttranslational farnesylation, and have been effective in treating Ras-dependent tumors in animal models (Sun J. et al. Oncogene 16: 1467-73, (1998)). In humans, clinical trials using a combination of anti-cancer drugs and an anti-HER2 monoclonal antibody, trastuzumab, to antagonize the proto-oncogene receptor HER2/neu, have improved clinical response and overall survival of a subset of breast-cancer patients (Molina M A, et.al. Cancer Res 61: 4744-9. (2001)). A tyrosine kinase inhibitor, STI-571, which selectively inactivates bcr-abl fusion proteins, has been developed to treat chronic myelogenous leukemias where constitutive activation of bcr-abl tyrosine kinase plays a crucial role in transformation of leukocytes. Agents of this kind are designed to suppress oncogenic activity of specific gene products (O'Dwyer M E, et al. Curr Opin Oncol 12: 594-7, (2000)). From the pharmacogenetic point of view, suppressing oncogenic signals is easier in practice than activating tumor-suppressive effects. Therefore, commonly up-regulated gene products, such as the MGC47816 and HES6 proteins, represent promising potential target for designing novel anti-cancer agents.

As demonstrated herein, suppressing the expression of MGC47816 and HES6 using short interfering RNA (siRNA) markedly decreased growth of cancer cells. Although the precise molecular mechanism by which the short interfering RNA (siRNA) can suppress growth needs to be clarified, the data herein clearly indicate that these genes are good candidates as diagnostic markers for HCC and may represent molecular targets for the development of effective drugs to treat this intractable tumor.

### INDUSTRIAL APPLICABILITY

The previous gene-expression analysis of genome-wide cDNA microarray identified MGC47816 and HES6 as specifically up-regulated genes. The present invention reveals that MGC47816 and HES6 also serve as targets for cancer prevention and therapy. Based on the expression of MGC47816 and/or HES6, the present invention provides a molecular diagnostic marker for identifying or detecting HCC.

The methods described herein are also useful in the identification of additional molecular targets for prevention, diagnosis and treatment of HCC. The data reported herein add to a comprehensive understanding of HCC, facilitate development of novel diagnostic strategies, and assist in the identification of molecular targets for therapeutic drugs and preventative agents. Such information contributes to a more profound understanding of hepatocellular tumorigenesis, and provides indicators for developing novel strategies for diagnosis, treatment, and ultimately prevention of HCC.

All patents, patent applications, and publications cited herein are incorporated by reference herein in their entirety. Furthermore, while the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention. Thus, the invention is intended to be defined not by the above description, but by the following claims and their equivalents.

1. A method of diagnosing HCC or a predisposition for developing HCC in a subject, comprising determining a level of expression of MGC47816 or HES6 in a patient-derived biological sample, wherein an increase in said sample expression level as compared to a normal control level of said gene indicates that said subject suffers from or is at risk of developing HCC.

2. The method of claim 1, wherein said sample expression level is at least 10% greater than said normal control level.

3. The method of claim 1, wherein the expression level is determined by any one method selected from group consisting of:
(a) detecting mRNA of MGC47816 or HES6,
(b) detecting a protein encoded by MGC47816 or HES6, and
(c) detecting a biological activity of a protein encoded by MGC47816 or HES6,

4. The method of claim 3, wherein said detection is carried out on a DNA array.

5. The method of claim 1, wherein said patient-derived biological sample comprises an epithelial cell.

6. The method of claim 1, wherein said patient-derived biological sample comprises a hepatocellular carcinoma cell.

7. The method of claim 1, wherein said patient-derived biological sample comprises an epithelial cell from a hepatocellular carcinoma.

8. A method of screening for a compound for treating or preventing HCC, said method comprising the steps of:
a) contacting a test compound with a polypeptide encoded by MGC47816 or HES6;
b) detecting the binding activity between the polypeptide and the test compound; and
c) selecting the test compound that binds to the polypeptide.

9. A method of screening for a compound for treating or preventing HCC, said method comprising the steps of:
a) contacting a candidate compound with a cell expressing MGC47816 or HES6, and
b) selecting the candidate compound that reduces the expression level of MGC47816 or HES6.

10. The method of claim 9, wherein said cell comprises a hepatocellular carcinoma cell.

11. A method of screening for a compound for treating or preventing HCC, said method comprising the steps of:
a) contacting a test compound with a polypeptide encoded by MGC47816 or HES6;
b) detecting the biological activity of the polypeptide of step (a); and
c) selecting the test compound that suppresses the biological activity of the polypeptide as compared to the biological activity detected in the absence of the test compound.

12. The method of claim 11, wherein the biological activity of the polypeptide is cell proliferative activity.

13. A method of screening for compound for treating or preventing HCC, said method comprising the steps of:
a) contacting a candidate compound with a cell into which a vector, comprising the transcriptional regulatory region of MGC47816 or HES6 and a reporter gene that is expressed under the control of the transcriptional regulatory region, has been introduced
b) measuring the expression or activity of said reporter gene; and
c) selecting the candidate compound that reduces the expression or activity of said reporter gene, as compared to a control.

14. A kit comprising a detection reagent which binds to (a) the nucleic acid sequence of MGC47816 or HES6 or (b) a polypeptide encoded thereby.

15. A method of treating or preventing HCC in a subject comprising administering to said subject an antisense composition, wherein said antisense composition comprises a nucleotide sequence complementary to a coding sequence of MGC47816 or HES6.

16. A method of treating or preventing HCC in a subject comprising administering to said subject an siRNA composition, wherein said siRNA composition reduces the expression of MGC47816 or HES6.

17. The method of claim 16, wherein the siRNA comprises a sense strand comprising a nucleotide sequence selected from the group consisting of of SEQ ID NO: 19 and 26 as the target sequence.

18. A method for treating or preventing HCC in a subject comprising the step of administering to said subject a pharmaceutically effective amount of an antibody, or fragment thereof, that binds to a protein encoded by MGC47816 or HES6.

19. A method of treating or preventing HCC in a subject comprising administering to said subject a vaccine comprising (a) a polypeptide encoded by MGC47816 or HES6, (b) an immunologically active fragment of said polypeptide, or (c) a polynucleotide encoding said polypeptide.

20. A method for treating or preventing HCC in a subject, said method comprising the step of administering a compound that is obtained by the method according to any one of claims 8-13.

21. A composition for treating or preventing HCC, said composition comprising a pharmaceutically effective amount of an antisense polynucleotide or small interfering RNA (siRNA) against MGC47816 or HES6 as an active ingredient, and a pharmaceutically acceptable carrier.

22. The composition of claim 21, wherein the siRNA comprises a sense strand comprising a nucleotide sequence selected from the group consisting of SEQ ID NO: 19 and 26 as the target sequence.

23. A composition for treating or preventing HCC, said composition comprising a pharmaceutically effective amount of an antibody or fragment thereof that binds to a protein encoded by MGC47816 or HES6 as an active ingredient, and a pharmaceutically acceptable carrier.

24. A composition for treating or preventing HCC, said composition comprising a pharmaceutically effective amount of a compound selected by the method of any one of claims 8-13 as an active ingredient, and a pharmaceutically acceptable carrier

25. A small interfering RNA, wherein the sense strand thereof comprises a nucleotide sequence selected from the group consisting of SEQ ID NO: 19 and 26 as the target sequence.


SEQUENCE LISTING (28 sequences)

SEQ ID NO: 1 - DNA, 1528 bp, Homo sapiens
  CDS (133)...(1308)
ccacgcgtccgcgggagcggagccgtggcgcgctcgccccggacgccggccgcccctccgctcgccctactgagcgagcggcccggggcgccgaggggtccgcgccgcgcggggcgcaccgccctggccgccatgtgctcccagctctggttcctgacggaccggcgcatccgcgaggactacccgcaggtgcagatcctgcgcgccctccggcagcgctgctccgagcaggacgtgcgcttccgggcggtgcttatggaccagatcgccgtcaccatcgtcggcggccacctcggcctccagctaaaccagaaggccctcaccactttcccggatgtggtgcttgtacgggtacccacaccctcagtgcagtcagacagtgacatcactgtcctgcgacacctggagaagctgggctgccggttggtcaatcgcccacagagcatcttaaattgcatcaacaaattctggacgttccaagaactggctggacatggggtccccatgccagacaccttctcctatggtgggcatgaagacttttcaaaaatgattgatgaagctgagcccctgggctacccagtcgtggtgaagagcacacgaggccaccggggaaaagctgtttttctggcaagagataaacatcacctctctgacatctgccatctgatccgccacgatgtgccctacctgttccagaagtacgtgaaggagtcccatggaaaggacatccgggtggtggtggtagggggccaggtcataggctctatgcttcgctgctccactgatggacggatgcagagcaactgctctctcggtggcgtgggcgtcaagtgtccgctgacagaacaaggcaagcagttggctattcaggtgtccaacatcctaggcatggacttctgtggcattgatctccttatcatggacgatggctcctttgtggtgtgtgaggcaaatgctaatgttggcttcctagcctttgaccaggcatgcaacttagatgtgggtgggatcattgcagactataccatgtccttgctgccaaataggcagactggaaagatggctgtcctcccaggactgtcgagtccaagggagaagaacgagccggatggctgtgcttcagctcagggagttgcagagagcgtctataccatcaacagtgggtctacctctagcgaaagtgagcctgaactgggagagatccgggattcctcagcaagcacaatgggggccccaccctccatgctgcccgaacctggctacaacattaacaacaggattgcttctgagttaaaacttaagtgaattcctgctttttggcagcatttaaaccaaatcctactgcttccctagtagttttgagtgaataaaatctggactaatgtgatttcatttgcacagaaactagaaatcccatctgggcactcagcattttttctaacgatgatttaagcaaatggcctagctttgtggtttttacaaagacaaatataaaaacactcacaagaacaaaaaaaaaaaaaaa

SEQ ID NO: 2 - protein, 391 aa, Homo sapiens
MCSQLWFLTDRRIREDYPQVQILRALRQRCSEQDVRFRAVLMDQIAVTIVGGHLGLQLNQKALTTFPDVVLVRVPTPSVQSDSDITVLRHLEKLGCRLVNRPQSILNCINKFWTFQELAGHGVPMPDTFSYGGHEDFSKMIDEAEPLGYPVVVKSTRGHRGKAVFLARDKHHLSDICHLIRHDVPYLFQKYVKESHGKDIRVVVVGGQVIGSMLRCSTDGRMQSNCSLGGVGVKCPLTEQGKQLAIQVSNILGMDFCGIDLLIMDDGSFVVCEANANVGFLAFDQACNLDVGGIIADYTMSLLPNRQTGKMAVLPGLSSPREKNEPDGCASAQGVAESVYTINSGSTSSESEPELGEIRDSSASTMGAPPSMLPEPGYNINNRIASELKLK

SEQ ID NO: 3 - DNA, 22 bp, Artificial Sequence
  An artificially synthesized primer sequence for RT-PCR
acaacagcctcaagatcatcag

SEQ ID NO: 4 - DNA, 20 bp, Artificial Sequence
  An artificially synthesized primer sequence for RT-PCR
ggtccaccactgacacgttg

SEQ ID NO: 5 - DNA, 23 bp, Artificial Sequence
  An artificially synthesized primer sequence for RT-PCR
caaataggcagactggaaagatg

SEQ ID NO: 6 - DNA, 23 bp, Artificial Sequence
  An artificially synthesized primer sequence for RT-PCR
ctagggaagcagtaggatttggt

SEQ ID NO: 7 - DNA, 30 bp, Artificial Sequence
  An artificially synthesized primer sequence for RT-PCR
attgtcgacgctcgccctactgagcgagcg

SEQ ID NO: 8 - DNA, 36 bp, Artificial Sequence
  An artificially synthesized primer sequence for RT-PCR
aatctcgagagcaggaattcacttaagttttaactc

SEQ ID NO: 9 - DNA, 22 bp, Artificial Sequence
  An artificially synthesized primer sequence for RT-PCR
tggtagccaagtgcaggttata

SEQ ID NO: 10 - DNA, 22 bp, Artificial Sequence
  An artificially synthesized primer sequence for RT-PCR
ccaaagggtttctgcagtttca

SEQ ID NO: 11 - DNA, 30 bp, Artificial Sequence
  An artificially synthesized primer sequence for RT-PCR
tgcggatccagagcagattgtactgagagt

SEQ ID NO: 12 - DNA, 29 bp, Artificial Sequence
  An artificially synthesized primer sequence for RT-PCR
ctctatctcgagtgaggcggaaagaacca

SEQ ID NO: 13 - DNA, 47 bp, Artificial Sequence
  An artificially synthesized primer sequence for RT-PCR
tttaagcttgaagaccatttttggaaaaaaaaaaaaaaaaaaaaaac

SEQ ID NO: 14 - DNA, 34 bp, Artificial Sequence
  An artificially synthesized primer sequence for RT-PCR
tttaagcttgaagacatgggaaagagtggtctca

SEQ ID NO: 15 - DNA, 51 bp, Artificial Sequence
  An artificially synthesized oligonucleotide sequence for siRNA
caccgaagcagcacgacttcttcttcaagagagaagaagtcgtgctgcttc

SEQ ID NO: 16 - DNA, 51 bp, Artificial Sequence
  An artificially synthesized oligonucleotide sequence for siRNA
aaaagaagcagcacgacttcttctctcttgaagaagaagtcgtgctgcttc

SEQ ID NO: 17 - DNA, 51 bp, Artificial Sequence
  An artificially synthesized oligonucleotide sequence for siRNA
tcccgtgtccgctgacagaacaattcaagagattgttctgtcagcggacac

SEQ ID NO: 18 - DNA, 51 bp, Artificial Sequence
  An artificially synthesized oligonucleotide sequence for siRNA
aaaagtgtccgctgacagaacaatctcttgaattgttctgtcagcggacac

SEQ ID NO: 19 - DNA, 19 bp, Artificial Sequence
  An artificially synthesized target sequence for siRNA
gtgtccgctgacagaacaa

SEQ ID NO: 20 - DNA, 21 bp, Artificial Sequence
  An artificially synthesized primer sequence for RT-PCR
gagctcctgaaccatctgctc

SEQ ID NO: 21 - DNA, 23 bp, Artificial Sequence
  An artificially synthesized primer sequence for RT-PCR
caagatgtacagagcatcacagc

SEQ ID NO: 22 - DNA, 29 bp, Artificial Sequence
  An artificially synthesized primer sequence for RT-PCR
attgaattcgcatggcgccacccgcggcg

SEQ ID NO: 23 - DNA, 32 bp, Artificial Sequence
  An artificially synthesized primer sequence for RT-PCR
aatggtacctcaccaaggcctccagacactcc

SEQ ID NO: 24 - DNA, 51 bp, Artificial Sequence
  An artificially synthesized oligonucletide sequence for siRNA
tcccacttttagggaccctgcagttcaagagactgcagggtccctaaaagt

SEQ ID NO: 25 - DNA, 51 bp, Artificial Sequence
  An artificially synthesized oligonucleotide sequence for siRNA
aaaaacttttagggaccctgcagtctcttgaactgcagggtccctaaaagt

SEQ ID NO: 26 - DNA, 19 bp, Artificial Sequence
  An artificially synthesized target sequence for siRNA
acttttagggaccctgcag

SEQ ID NO: 27 - DNA, 1375 bp, Homo sapiens
  CDS (125)...(799)
ggagcgcggacggctgggctgctgctgggcggccgcggggcagcggagggcgccggcactccggtccccgccgctccccgtccccgctgctcctagcccctgccgcgtccccggcggagcgggcatggcgccacccgcggcgcctggccgggaccgtgtgggccgtgaggatgaggacggctgggagacgcgaggggaccgcaaggcccggaagcccctggtggagaagaagcggcgcgcgcggatcaacgagagcctgcaggagctgcggctgctgctggcgggcgccgaggtgcaggccaagctggagaacgccgaagtgctggagctgacggtgcggcgggtccagggtgtgctgcggggccgggcgcgcgagcgcgagcagctgcaggcggaagcgagcgagcgcttcgctgccggctacatccagtgcatgcacgaggtgcacacgttcgtgtccacgtgccaggccatcgacgctaccgtcgctgccgagctcctgaaccatctgctcgagtccatgccgctgcgtgagggcagcagcttccaggatctgctgggggacgccctggcggggccacctagagcccctggacggagtggctggcctgcggggggcgctccgggatccccaatacccagccccccgggtcctggggacgacctgtgctccgacctggaggaggcccctgaggctgaactgagtcaggctcctgctgaggggcccgacttggtgcccgcagccctgggcagcctgaccacagcccaaattgcccggagtgtctggaggccttggtgaccaatgccagccagagtcctgcgggggtgggcccggccctccctggatctcctccctcctcccaggggttcagatgtggtggggtagggccctggaagtctcccaggtcttccctccctcctctgatggatggcttgcagggcagcccctggtaaccagcccagtcaggccccagccccgtttcttaagaaacttttagggaccctgcagctctggagtgggtggagggagggagctacgggcaggaggaagaattttgtagagctgccagcgctctcccaggttcacccacccagccttcaccagccctgtgcgggctctgggggcagaggtggcaggaatggtgctgggcactagtgttccaggcagccctgggctaaacaaaagcttgaacttgccacttcagcggggagatgagaggcaggtgcactcagctgcactgcccagagctgtgatgctctgtacatcttgtttgtagcacacttgagtttgtgtattccattgacatcaaatgtgacaattttactaaataaagaattttggagttagttacccttgaaaaaaaaaaaaaaaaaa

SEQ ID NO: 28 - protein, 224 aa, Homo sapiens
MAPPAAPGRDRVGREDEDGWETRGDRKARKPLVEKKRRARINESLQELRLLLAGAEVQAKLENAEVLELTVRRVQGVLRGRAREREQLQAEASERFAAGYIQCMHEVHTFVSTCQAIDATVAAELLNHLLESMPLREGSSFQDLLGDALAGPPRAPGRSGWPAGGAPGSPIPSPPGPGDDLCSDLEEAPEAELSQAPAEGPDLVPAALGSLTTAQIARSVWRPW